(19) United States
(12) Patent Application Publication
(10) Pub. No.: US 2007/0232654 A1
(43) Pub. Date: Oct. 4, 2007

(54) Novel Compounds and Compositions as Cathepsin Inhibitors

(75) Inventors: John W. Patterson, Mountain View, CA (US); Sheila Zipfel, Mountain View, CA (US)

Correspondence Address: SYNNESTVEDT & LECHNER LLP, 2600 ARAMARK TOWER, 1101 MARKET STREET, PHILADELPHIA, PA, 19107-2950, US

(73) Assignee: Axys Pharmaceuticals, Inc., South San Francisco, CA (US)

(21) Appl. No.: 11/758,894

(22) Filed: Jun. 6, 2007

Related U.S. Application Data
(62) Division of application No. 10/478,632, filed on Nov. 24, 2003.
(60) Provisional application No. 60/295,744, filed on Jun. 4, 2001.

(51) Int. Cl. A61K 31/44; A61K 31/16; C07C 213/00; C07D 263/52; C07C 233/00; A61K 31/423

(52) U.S. Cl. 514/317; 514/375; 514/616; 546/252; 548/217; 564/155

(57) ABSTRACT

The present invention relates to novel selective cathepsin S inhibitors, the pharmaceutically acceptable salts and N-oxides thereof, their uses as therapeutic agents and the methods of their making.

### CROSS-REFERENCE TO RELATED APPLICATIONS

This Application is a divisional of Non-Provisional application Ser. No. 10/478,632, filed on Nov. 24, 2003, which is a National Stage application of PCT Application No. PCT/US02/17922, filed Jun. 4, 2002, which claims priority benefit of Provisional Application No. 60/295,744, filed on Jun. 4, 2001. The disclosures of all three applications are incorporated by reference.

### THE INVENTION

This Application relates to compounds and compositions for treating diseases associated with cysteine protease activity, particularly diseases associated with activity of cathepsin S.

### DESCRIPTION OF THE FIELD

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increase expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy and others. An increase in cathepsin S activity contributes to the pathology and/or symptomatology of a number of diseases. Accordingly, molecules that inhibit the activity of cathepsin S protease are useful as therapeutic agents in the treatment of such diseases.

### SUMMARY OF THE INVENTION

This Application relates to compounds of Formula I: 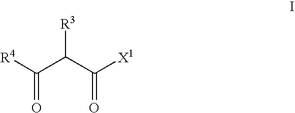
in which:

X¹ is —NHC(R¹)(R²)X² or —NHX³;

X² is cyano, —C(R⁷)(R⁸)X³, —C(R⁷)(R⁸)CF₃, —C(R⁷)(R⁸)CF₂CF₂R⁹—CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is (C₁₋₄)alkyl, (C₅₋₁₀)aryl(C₀₋₆)alkyl or (C₅₋₁₀)heteroaryl(C₀₋₆)alkyl; R⁶ is hydrogen or (C₁₋₆)alkyl; R⁷ is hydrogen or (C₁₋₄)alkyl and R⁸ is hydroxy or R⁷ and R⁸ together form oxo; R⁹ is hydrogen, halo, (C₁₋₄)alkyl, (C₅₋₁₀)aryl(C₀₋₆)alkyl or (C₅₋₁₀)heteroaryl(C₀₋₆)alkyl;

X³ comprises a heteromonocyclic ring containing 4 to 6 ring member atoms or a fused heterobicyclic ring system containing 8 to 14 ring member atoms and any carbocyclic ketone, iminoketone or thioketone derivative thereof;

wherein within R⁵, X² or X³ any alicyclic or aromatic ring system may be substituted further by 1 to 5 radicals independently selected from (C₁₋₆)alkyl, (C₁₋₆)alkylidene, cyano, halo, halo-substituted(C₁₋₄)alkyl, nitro, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³ and —X⁴S(O)₂R¹³ and/or 1 radical selected from —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴C(O)R¹⁴, —X⁴NR¹⁴R¹², —X⁴NR¹²C(O)R¹³, —X⁴NR¹²C(O)OR¹⁴, —X⁴C(O)NR¹⁴R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹², wherein X⁴ is a bond or (C₁₋₆)alkyl; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹ is hydrogen, halo or (C₁₋₆)alkyl and R² is selected from a group consisting of hydrogen, cyano, halo, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³, —X⁴S(O)₂R¹³, —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴OC(O)R¹⁴, —X⁴NR¹⁴R¹², —X⁴NR¹⁴C(O)R¹⁴, —X⁴NR¹²C(O)OR¹⁴, —X⁴C(O)NR¹²R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹², wherein X⁴, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is optionally substituted with 1 to 3 radicals independently selected from (C₁₋₆)alkyl, (C₁₋₆)alkylidene, cyano, halo, halo-substituted(C₁₋₄)alkyl, nitro, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂N¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³, —X⁴S(O)₂R¹³ and —X⁴C(O)R¹³, wherein X⁴, R¹² and R¹³ are as defined above;

R³ is —C(R⁶)(R⁶)X⁵, wherein R⁶ is as defined above and X⁵ is selected from —XNR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴R¹², —X⁴OP(O)(OR¹²)OR¹², —X⁴C(O)R¹³, —X⁴NR¹²C(O)R¹³, —X⁴S(O)R¹³ and —X⁴S(O)₂R¹³, —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴C(O)R¹⁴, —X⁴NR¹⁴R¹², —X⁴NR¹²C(O)R¹⁴, —X⁴NR¹²C(O)OR¹⁴, —X⁴C(O)NR¹⁴R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹² wherein X⁴, R¹², R¹³ and R¹⁴ are as defined above;

R⁴ is —NR⁶R⁶, —NR⁶R¹⁴, —NR⁶R¹⁵ or —NR⁶X⁵C(O)R¹⁴ wherein R⁶, X⁵ and R¹⁴ are as described above and R¹⁵ is hydrogen, —(C₁₋₆)alkyl or —X⁵OR⁶ wherein X⁵ is as described above; or R⁶ and R¹⁵ together with the nitrogen atom to which R⁶ and R¹⁵ are attached form hetero(C₃₋₁₀)cycloalkyl, hetero(C₅₋₁₀)aryl or hetero(C₈₋₁₀)bicycloaryl;

wherein within R³ and R⁴ any alicyclic or aromatic ring system may be substituted further by 1-5 radicals independently selected from (C₁₋₆)alkyl, (C₁₋₆)alkylidene, cyano, halo, halo-substituted(C₁₋₄)alkyl, nitro, —X⁴NR¹²R¹², —X⁴NR¹²C(O)R¹², —X⁴NR¹²C(O)R¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴ OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³, —X⁴C(O)R¹³ and —X⁴S(O)₂R¹³ and/or 1 radical selected from —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴OC(O)R¹⁴, —X⁴NR¹⁴R¹², —X⁴NR¹²C(O)R¹⁴, —X⁴NR¹²C(O)OR¹⁴, —X⁴C(O)NR¹⁴R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹²; and within R³ and R⁴ any aliphatic moiety may be substituted further by 1-5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁴, R¹², R¹³ and R¹⁴ are as described above;

with the proviso that only one bicyclic ring structure is present within R³ or R⁴; and the N-oxide derivatives, prodrug derivatives, protected derivatives, individual isomers and mixtures of isomers thereof, and the pharmaceutically acceptable salts and solvates (e.g. hydrates) of such compounds and the N-oxide derivatives, prodrug derivatives, protected derivatives, individual isomers and mixtures of isomers thereof.

A second aspect of the invention is a pharmaceutical composition which contains a compound of Formula I or a N-oxide derivative, individual isomer or mixture of isomers thereof, or a pharmaceutically acceptable salt thereof, in admixture with one or more suitable excipients.

A third aspect of the invention is a method for treating a disease in an animal in which inhibition of cathepsin S can prevent, inhibit or ameliorate the pathology and/or symptomatology of the disease, which method comprises administering to the animal a therapeutically effective amount of compound of Formula I or a N-oxide derivative, individual isomer or mixture of isomers thereof, or a pharmaceutically acceptable salt thereof.

A fourth aspect of the invention is the processes for preparing compounds of Formula I and the N-oxide derivatives, prodrug derivatives, protected derivatives, individual isomers and mixtures of isomers thereof, and the pharmaceutically acceptable salts thereof.

### DETAILED DESCRIPTION OF THE INVENTION

Definitions:

Unless otherwise stated, the following terms used in the specification and claims are defined for the purposes of this Application and have the following meanings.

“Alicyclic” means a moiety characterized by arrangement of the carbon atoms in closed non-aromatic ring structures having properties resembling those of aliphatics and may be saturated or partially unsaturated with two or more double or triple bonds.

“Aliphatic” means a moiety characterized by a straight or branched chain arrangement of the constituent carbon atoms and may be saturated or partially unsaturated with two or more double or triple bonds.

“Alkyl” represented by itself means a straight or branched, saturated or unsaturated, aliphatic radical having the number of carbon atoms indicated (e.g., (C₁₋₆)alkyl includes methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl) means a straight or branched, saturated or unsaturated aliphatic divalent radical having the number of atoms indicated or when no atoms are indicated means a bond (e.g., (C₆₋₁₀)aryl(C₀₋₃)alkyl includes phenyl, benzyl, phenethyl, 1-phenylethyl 3-phenylpropyl, and the like).

“Alkylene”, unless indicated otherwise, means a straight or branched, saturated or unsaturated, aliphatic, divalent radical having the number of carbon atoms indicated (e.g., (C₁₋₆)alkylene includes methylene (—CH₂—), ethylene (—CH₂CH₂—), trimethylene (—CH₂CH₂CH₂—), tetramethylene (—CH₂CH₂CH₂CH₂—) 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like).

“Alkylidene” means a straight or branched saturated or unsaturated, aliphatic, divalent radical having the number of carbon atoms indicated (e.g. (C₁₋₆)alkylidene includes methylidene (═CH₂), ethylidene (═CHCH₃), isopropylidene (═C(CH₃)₂), propylidene (═CHCH₂CH₃), allylidene (═CH⁻CH═CH₂), and the like).

“Amino” means the radical —NH₂. Unless indicated otherwise, the compounds of the invention containing amino moieties include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2.

“Aryl” means a monocyclic or fused bicyclic ring assembly containing the total number of ring carbon atoms indicated, wherein each ring is comprised of 6 ring carbon atoms and is aromatic or when fused with a second ring forms an aromatic ring assembly. For example, optionally substituted (C₆₋₁₀)aryl as used in this Application includes, but is not limited to, biphenyl-2-yl, 2-bromophenyl, 2-bromocarbonylphenyl, 2-bromo-5-fluorophenyl, 4-tert-butylphenyl, 4-carbamoylphenyl, 4-carboxy-2-nitrophenyl, 2-chlorophenyl, 4-chlorophenyl, 3-chlorocarbonylphenyl, 4-chlorocarbonylphenyl, 2-chloro-4-fluorophenyl, 2-chloro-6-fluorophenyl, 4-chloro-2-nitrophenyl, 6-chloro-2-nitrophenyl, 2,6-dibromophenyl, 2,3-dichlorophenyl, 2,5-dichlorophenyl, 3,4-dichlorophenyl, 2-difluoromethoxyphenyl, 3,5-dimethylphenyl, 2-ethoxycarbonylphenyl, 2-fluorophenyl, 2-iodophenyl, 4-isopropylphenyl, 2-methoxyphenyl, 4-methoxyphenyl, 2-methylphenyl, 3-methylphenyl, 4-methylphenyl, 5-methyl-2-nitrophenyl, 4-methylsulfonylphenyl, naphth-2-yl, 2-nitrophenyl, 3-nitrophenyl, 4-nitrophenyl, 2,3,4,5,6-pentafluorophenyl, phenyl, 2-trifluoromethoxyphenyl, 3-trifluoromethoxyphenyl, 4-trifluoromethoxyphenyl, 2-trifluoromethylphenyl, 3-trifluoromethylphenyl, 4-trifluoromethylphenyl, 2-trifluoromethylsulfanylphenyl, 4-trifluoromethylsulfanylphenyl, and the like. Optionally substituted (C₆₋₁₀)aryl as used in this Application includes 3-acetylphenyl, 3-tert-butoxycarbonylaminomethylphenyl, biphenyl-4-yl, 3-hydroxyphenyl, 4-hydroxyphenyl, 3-methoxyphenyl, naphth-2-yl, 3-phenoxyphenyl, phenyl, and the like.

“Bicycloaryl” means a bicyclic ring assembly containing the number of ring carbon atoms indicated, wherein the rings are linked by a single bond or fused and at least one of the rings comprising the assembly is aromatic, and any carbocyclic ketone, thioketone or iminoketone derivative thereof (e.g., (C₉₋₁₀)bicycloaryl includes cyclohexylphenyl, 1,2-dihydronaphthyl, 2,4-dioxo-1,2,3,4-tetrahydronaphthyl, indanyl, indenyl, 1,2,3,4-tetrahydronaphthyl, and the like).

“Carbamoyl” means the radical —C(O)NH₂. Unless indicated otherwise, the compounds of the invention containing carbamoyl moieties include protected derivatives thereof. Suitable protecting groups for carbamoyl moieties include acetyl, tert-butoxycarbonyl, benzyloxycarbonyl, and the like and both the unprotected and protected derivatives fall within the scope of the invention.

“Carbocyclic ketone derivative” means a derivative containing the moiety —C(O)—.

“Carboxy” means the radical —C(O)OH. Unless indicated otherwise, the compounds of the invention containing carboxy moieties include protected derivatives thereof. Suitable protecting groups for carboxy moieties include benzyl, tert-butyl, and the like.

“Cycloalkyl” means a saturated or partially unsaturated, monocyclic, fused bicyclic or bridged polycyclic ring assembly containing the number of ring carbon atoms indicated, and any carbocyclic ketone, thioketone or iminoketone derivative thereof (e.g., (C₃₋₁₀)cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like).

“Cycloalkylene” means a divalent saturated or partially unsaturated, monocyclic ring or bridged polycyclic ring assembly containing the number of ring carbon atoms indicated, and any carbocyclic ketone, thioketone or iminoketone derivative thereof. For example, the instance wherein “R¹ and R² together with the carbon atom to which both R¹ and R² are attached form (C₃₋₈)cycloalkylene” includes, but is not limited to, the following:

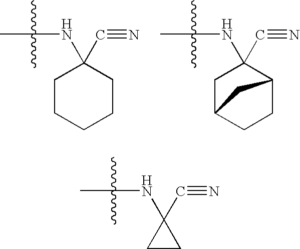

“Disease” specifically includes any unhealthy condition of an animal or part thereof and includes an unhealthy condition that may be caused by, or incident to, medical or veterinary therapy applied to that animal, i.e., the “side effects” of such therapy.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like (e.g. halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroatom moiety” includes —N═, —NR—, —O—, —S— or —S(O)₂—, wherein R is hydrogen, (C₁₋₆)alkyl or a protecting group.

“Heterocycloalkylene” means cycloalkylene, as defined in this Application, provided that one or more of the ring member carbon atoms indicated, is replaced by heteroatom moiety selected from —N═, —NR—, —O—, —S— or —S(O)₂—, wherein R is hydrogen or (C₁₋₆)alkyl. For example, the instance wherein R¹ and R² together with the carbon atom to which both R¹ and R² are attached form hetero(C₃₋₈)cycloalkyl” includes, but is not limited to, the following: 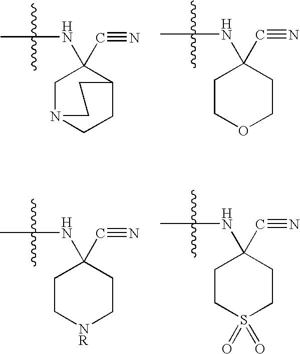
in which R is hydrogen, (C₁₋₆)alkyl, or a protecting group.

“Heteroaryl” means aryl, as defined in this Application, provided that one or more of the ring carbon atoms indicated are replaced by a heteroatom moiety selected from —N═, —NR—, —O— or —S—, wherein R is hydrogen, (C₁₋₆)alkyl, a protecting group or represents the free valence which serves as the point of attachment to a ring nitrogen, and each ring is comprised of 5 or 6 ring atoms. For example, optionally substituted hetero(C₅₋₁₀)aryl as used in this Application includes, but is not limited to, 4-amino-2-hydroxypyrimidin-5-yl, benzothiazol-2-yl, 1H-benzoimidazol-2-yl, 2-bromopyrid-5-yl, 5-bromopyrid-2-yl, 4-carbamoylthiazol-2-yl, 3-carboxypyrid-4-yl, 5-carboxy-2,6-dimethylpyrid-3-yl, 3,5-dimethylisoxazol-4-yl, 5-ethoxy-2,6-dimethylpyrid-3-yl, 5-fluoro-6-hydroxypyrimidin-4-yl, fur-2-yl, fur-3-yl, 5-hydroxy-4,6-dimethylpyrid-3-yl, 8-hydroxy-5,7-dimethylquinolin-2-yl, 5-hydroxymethylisoxazol-3-yl, 3-hydroxy-6-methylpyrid-2-yl, 3-hydroxypyrid-2-yl, 1H-imidazol-2-yl, 1H-imidazol-4-yl, 1H-indol-3-yl, isothiazol-4-yl, isoxazol-4-yl, 2-methylfur-3-yl, 5-methylfur-2-yl, 1-methyl-1H-imidazol-2-yl, 5-methyl-3H-imidazol-4-yl, 5-methylisoxazol-3-yl, 5-methyl-2H-pyrazol-3-yl, 3-methylpyrid-2-yl, 4-methylpyrid-2-yl, 5-methylpyrid-2-yl, 6-methylpyrid-2-yl, 2-methylpyrid-3-yl, 2-methylthiazol-4-yl, 5-nitropyrid-2-yl, 2H-pyrazol-3-yl, 3H-pyrazol-4-yl, pyridazin-3-yl, pyrid-2-yl, pyrid-3-yl, pyrid-4-yl, 5-pyrid-3-yl-2H-[1,2,4]triazol-3-yl, pyrimidin-4-yl, pyrimidin-5-yl, 1H-pyrrol-3-yl, quinolin-2-yl, 1H-tetrazol-5-yl, thiazol-2-yl, thiazol-5-yl, thien-2-yl, thien-3-yl, 2H-[1,2,4]triazol-3-yl, 3H-[1,2,3]triazol-4-yl, 5-trifluoromethylpyrid-2-yl, and the like. Suitable protecting groups include tert-butoxycarbonyl, benzyloxycarbonyl, benzyl, 4-methoxybenzyl, 2-nitrobenzyl, and the like. Optionally substituted hetero(C₅₋₁₀)aryl as used in this Application to define R⁴ includes benzofur-2-yl, fur-2-yl, fur-3-yl, pyrid-3-yl, pyrid-4-yl, quinol-2-yl, quinol-3-yl, thien-2-yl, thien-3-yl, and the like.

“Heterobicycloaryl” means bicycloaryl, as defined in this Application, provided that one or more of the ring carbon atoms indicated are replaced by a heteroatom moiety selected from —N═, —NR—, —O— or —S—, wherein R is hydrogen, (C₁₋₆)alkyl, a protecting group or represents the free valence which serves as the point of attachment to a ring nitrogen, and any carbocyclic ketone, thioketone or iminoketone derivative thereof. For example, optionally substituted hetero(C₈₋₁₀)bicycloaryl as used in this Application includes, but is not limited to, 2-amino-4-oxo-3,4-dihydropteridin-6-yl, and the like. In general, the term heterobicycloaryl as used in this Application includes, for example, benzo[1,3]dioxol-5-yl, 3,4-dihydro-2H-[1,8]naphthyridinyl, 3,4-dihydro-2H-quinolinyl, 2,4-dioxo-3,4-dihydro-2H-quinazolinyl, 1,2,3,4,5,6-hexahydro[2,2′]bipyridinylyl, 3-oxo-2,3-dihydrobenzo[1,4]oxazinyl, 5,6,7,8-tetrahydroquinolinyl, and the like.

“Heterocycloalkyl” means cycloalkyl, as defined in this Application, provided that one or more of the ring carbon atoms indicated are replaced by a heteroatom moiety selected from —N═, —NR—, —O— or —S—, wherein R is hydrogen, (C₁₋₆)alkyl, a protecting group or represents the free valence which serves as the point of attachment to a ring nitrogen, and any carbocyclic ketone, thioketone or iminoketone derivative thereof (e.g., the term hetero(C₅₋₁₀)cycloalkyl includes imidazolidinyl, morpholinyl, piperazinyl, piperidyl, pyrrolidinyl, pyrrolinyl, quinuclidinyl, and the like). Suitable protecting groups include tert-butoxycarbonyl, benzyloxycarbonyl, benzyl, 4-methoxybenzyl, 2-nitrobenzyl, and the like. Both the unprotected and protected derivatives fall within the scope of the invention.

“Hydroxy” means the radical —OH. Unless indicated otherwise, the compounds of the invention containing hydroxy radicals include protected derivatives thereof. Suitable protecting groups for hydroxy moieties include benzyl and the like.

“Iminoketone derivative” means a derivative containing the moiety —C(NR)—, wherein R is hydrogen or (C₁₋₆)alkyl.

“Isomers” mean compounds of Formula I having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula I are meant to be encompassed all possible stereoisomers. Thus, for example, the name N-[1-(1-Benzylcarbamoyl-methanoyl)-propyl]-4-morpholin-4-yl-4-oxo-2-phenylmethanesulfonylmethyl-butyramide is meant to include N-[(S)-1-(1-Benzylcarbamoyl-methanoyl)-propyl]-4-morpholin-4-yl-4-oxo-2-phenylmethanesulfonylmethyl-butyramide and N-[(R)-1-(1-Benzylcarbamoyl-methanoyl)-propyl]-4-morpholin-4-yl-4-oxo-2-phenylmethanesulfonylmethyl-butyramide and any mixture, racemic or otherwise, thereof.

“Ketone derivative” means a derivative containing the moiety —C(O)—. For example, in this Application X³ can be 2-acetoxy-azetidin-3-yl. The “carbocyclic ketone derivative” of this example of X³ would be 2-acetoxy-4-oxo-azetidin-3-yl (see Table 3, C32).

“Nitro” means the radical —NO₂.

“Optional” or “optionally” means that the subsequently described event or circumstance may or may not occur, and that the description includes instances where the event or circumstance occurs and instances in which it does not. For example, the phrase “wherein within R³ and R⁴ any alicyclic or aromatic ring system may be substituted further by 1-5 radicals . . . ” means that R³ and R⁴ may or may not be substituted in order to fall within the scope of the invention.

“Oxoalkyl” means alkyl, as defined above, wherein one of the number of carbon atoms indicated is replaced by an oxygen group (—O—), e.g., oxo(C₂₋₆)alkyl includes methoxymethyl, etc.

“N-oxide derivatives” means derivatives of compounds of Formula I in which nitrogens are in an oxidized state (i.e., O—N) and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula I which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartatic acid, citric acid, benzoic acid, o-(4-hydroxybenzoyl)benzoic acid, cinnamic acid, madelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound which is convertible in vivo by metabolic means (e.g. by hydrolysis) to a compound of Formula I. For example an ester of a compound of Formula I containing a hydroxy group may be convertible by hydrolysis in vivo to the parent molecule. Alternatively an ester of a compound of Formula I containing a carboxy group may be convertible by hydrolysis in vivo to the parent molecule. Suitable esters of compounds of Formula I containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulphonates, ethanesulphonates, benzenesulphonates, p-toluenesulphonates, cyclohexylsulphamates and quinates. Suitable esters of compounds of Formula I containing a carboxy group, are for example those described by F. J. Leinweber, Drug Metab. Res., 1987, 18, page 3 79. An especially useful class of esters of compounds of Formula I containing a hydroxy group, may be formed from acid moieties selected from those described by Bundgaard et al., J. Med. Chem., 1989, 32, page 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)-benzoates.

“Protected derivatives” means derivatives of compounds of Formula I in which a reactive site or sites are blocked with protecting groups. Protected derivatives of compounds of Formula I are useful in the preparation of compounds of Formula I or in themselves may be active cathepsin S inhibitors. A comprehensive list of suitable protecting groups can be found in T. W. Greene, Protecting Groups in Organic Synthesis, 3rd edition, John Wiley & Sons, Inc. 1999.

“Therapeutically effective amount” means that amount which, when administered to an animal for treating a disease, is sufficient to effect such treatment for the disease.

“Thioketone derivative” means a derivative containing the moiety —C(S)—.

“Treatment” or “treating” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).
   Nomenclature:

The compounds of Formula I and the intermediates and starting materials used in their preparation are named in accordance with IUPAC rules of nomenclature in which the characteristic groups have decreasing priority for citation as the principle group as follows: acids, esters, amides, etc. Alternatively, the compounds are named by AutoNom 4.0 (Beilstein Information Systems, Inc.). For example, a compound of Formula I in which X¹ is —NHC(R¹)(R²)X² (R¹ and R² are each hydrogen), X² is cyano, R³ is cyclohexylmethyl, and R⁴ is phenylamino; that is, a compound having the following structure: 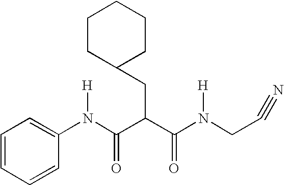
is named N-cyanomethyl-2-cyclohexylmethyl-N′-phenyl-malonamide.

### Presently Preferred Embodiments

While the broadest definition of the invention is set forth in the Summary of the Invention, certain aspects of the invention are preferred. For example, X¹ is —NHC(R¹)(R²)X² or —NHX³; X² is cyano, —C(O)X³, —C(O)CF₃, —C(O)CF₂CF₂R⁹, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ and R⁶ are as described above; X³ comprises a heteromonocyclic ring containing 4 to 6 ring member atoms or a fused heterobicyclic ring system containing 8 to 14 ring member atoms and any carbocyclic ketone, iminoketone or thioketone derivative thereof, wherein within R⁵, X² or X³ any alicyclic or aromatic ring system may be substituted further by 1 to 5 radicals independently selected from (C₁₋₆)alkyl or —X⁴OC(O)R¹² and/or 1 radical selected from —R¹⁴, —X⁴C(O)R¹⁴ or —X⁴OC(O)R¹⁴; wherein X⁴, R¹² and R¹⁴ are as described above; R¹ is hydrogen or (C₁₋₆)alkyl and R² is hydrogen, —X⁴OR¹², (C₅₋₁₀)heteroaryl(C₀₋₆)alkyl, (C₅₋₁₀)aryl(C₀₋₆)alkyl, (C₅₋₁₀)cycloalkyl(C₀₋₆)alkyl, (C₅₋₁₀)heterocycloalkyl(C₀₋₆)alkyl or (C₁₋₆)alkyl; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is optionally substituted with 1 to 3 radicals independently selected from (C₁₋₆)alkyl and hydroxy; R³ is —CH₂X⁵, wherein X⁵ at each occurrence independently is selected from —X⁴SR¹², —X⁴C(O)NR¹²R¹², —X⁴S(O)₂R¹³, —X⁴C(O)R¹³, —X⁴SR¹⁴, —X⁴R¹², —R¹⁴, —X⁴S(O)₂R¹⁴, —X⁴C(O)R¹⁴, —X⁴C(O)NR¹⁴R¹², wherein X⁴, R¹², R¹³ and R¹⁴ are as defined above; R⁴ is —NR⁶R⁶, —NR⁶R¹⁴, —NR⁶R¹⁵ or —NR⁶X⁵C(O)R¹⁴ wherein R⁶, X⁵ and R¹⁴ are as described above and R¹⁵ is hydrogen, —(C₁₋₆)alkyl or —X⁵OR⁶ wherein X⁵ is as described above; or R⁶ and R¹⁵ together with the nitrogen atom to which R⁶ and R¹⁵ are attached form hetero(C₃₋₁₀)cycloalkyl, hetero(C₅₋₁₀)aryl or hetero(C₈₋₁₀)bicycloaryl; wherein within R³ and R⁴ any alicyclic or aromatic ring system may be substituted further by 1-5 radicals independently selected from (C₁₋₆)alkyl, cyano, halo, nitro, halo-substituted(C₁₋₄)alkyl, —X⁴OR¹², —X⁴C(O)OR¹², —X⁴C(O)R¹³, —X⁴C(O)NR¹²R¹², —X⁴NR¹²S(O)₂R¹² and/or 1 radical selected from —R¹⁴, —X⁴OR¹⁴ and —X⁴C(O)NR¹⁴R¹²; within R³ and R⁴ any aliphatic moiety may be substituted further by 1-5 radicals independently selected from cyano; wherein X⁴, R¹², R¹³ and R¹⁴ are as described above; with the proviso that only one bicyclic ring structure is present within R³ or R⁴.

In particular, X¹ is —NHC(R¹)(R²)X² or —NHX³; X² is cyano, —C(O)X³, —CF₃, —CF₂CF₃, (E)-2-benzenesulfonyl-vinyl, 2-dimethylcarbamoyl-2,2-difluoro-acetyl, 1-benzylcarbamoyl-methanoyl, 1-benzyloxy(oxalyl), 2-benzyloxy-acetyl, 2-benzenesulfonylamino-ethanoyl or 2-oxo-2-phenyl-ethanoyl; X³ is 1H-benzoimidazol-2-yl, pyrimidin-2-yl, benzooxazol-2-yl, benzothiazol-2-yl, pyridazin-3-yl, 3-phenyl-[1,2,4]oxadiazol-5-yl, 3-ethyl-[1,2,4]oxadiazol-5-yl, 2-methyl-4-oxo-tetrahydro-furan-3-yl, 2-ethyl-4-oxo-tetrahydro-furan-3-yl, 4-oxo-1-(1-phenyl-methanoyl)-pyrrolidin-3-yl or (S)-2-Acetoxy-4-oxo-azetidin-3-yl; R¹ is hydrogen or methyl and R² is hydrogen, methoxymethyl, (C₁₋₆)alkyl, phenethyl, thiophen-2-yl or 5-methyl-furan-2-yl, or (ii) R¹ and R² taken together with the carbon atom to which both R¹ and R² are attached form cyclopropylene, tetrahydro-pyran-4-ylene or methyl-piperidin-4-ylene.

R³ more preferably is thiophene-2-sulfonylmethyl, 3-chloro-2-fluoro-phenylmethanesulfonylmethyl, benzenesulfonylmethyl, phenylmethanesulfonylmethyl, 2-(1,1-difluoro-methoxy)-phenylmethanesulfonylmethyl, 2-benzenesulfonyl-ethyl, 2-(pyridine-2-sulfonyl)-ethyl, 2-(pyridine-4-sulfonyl)-ethyl, 2-phenylmethanesulfonyl-ethyl, oxy-pyridin-2-ylmethanesulfonylmethyl, prop-2-ene-1-sulfonylmethyl, 4-methoxy-phenylmethanesulfonylmethyl, p-tolylmethanesulfonylmethyl, 4-chloro-phenylmethanesulfonylmethyl, o-tolylmethanesulfonylmethyl, 3,5-dimethyl-phenylmethanesulfonylmethyl, 4-trifluoromethyl-phenylmethanesulfonylmethyl, 4-trifluoromethoxy-phenylmethanesulfonylmethyl, 2-bromo-phenylmethanesulfonylmethyl, pyridin-2-ylmethanesulfonylmethyl, pyridin-3-ylmethanesulfonylmethyl, pyridin-4-ylmethanesulfonylmethyl, naphthalen-2-ylmethanesulfonylmethyl, 3-methyl-phenylmethanesulfonylmethyl, 3-trifluoromethyl-phenylmethanesulfonylmethyl, 3-trifluoromethoxy-phenylmethanesulfonylmethyl, 4-fluoro-2-trifluoromethoxy-phenylmethanesulfonylmethyl, 2-fluoro-6-trifluoromethyl-phenylmethanesulfonylmethyl, 3-chloro-phenylmethanesulfonylmethyl, 2-fluoro-phenylmethanesulfonylmethyl, 2-trifluoro-phenylmethanesulfonylmethyl, 2-cyano-phenylmethanesulfonylmethyl, 4-tert-butyl-phenylmethanesulfonylmethyl, 2-fluoro-3-methyl-phenylmethanesulfonylmethyl, 3-fluoro-phenylmethanesulfonylmethyl, 4-fluoro-phenylmethanesulfonylmethyl, 2-chloro-phenylmethanesulfonylmethyl, 2,5-difluoro-phenylmethanesulfonylmethyl, 2,6-difluoro-phenylmethanesulfonylmethyl, 2,5-dichloro-phenylmethanesulfonylmethyl, 3,4-dichloro-phenylmethanesulfonylmethyl, 2-(1,1-difluoro-methoxy)-phenylmethanesulfonylmethyl, 2-cyano-phenylmethanesulfonylmethyl, 3-cyano-phenylmethanesulfonylmethyl, 2-trifluoromethoxy-phenylmethanesulfonylmethyl, 2,3-difluoro-phenylmethanesulfonylmethyl, 2,5-difluoro-phenylmethanesulfonylmethyl, biphenyl-2-ylmethanesulfonylmethyl, cyclohexylmethyl, 3-fluoro-phenylmethanesulfonylmethyl, 3,4-difluoro-phenylmethanesulfonylmethyl, 2,4-difluoro-phenylmethanesulfonylmethyl, 2,4,6-trifluoro-phenylmethanesulfonylmethyl, 2,4,5-trifluoro-phenylmethanesulfonylmethyl, 2,3,4-trifluoro-phenylmethanesulfonylmethyl, 2,3,5-trifluoro-phenylmethanesulfonylmethyl, 2,5,6-trifluoro-phenylmethanesulfonylmethyl, 2-chloro-5-trifluoromethylphenylmethanesulfonylmethyl, 2-methyl-propane-1-sulfonyl, 2-fluoro-3-trifluoromethylphenylmethanesulfonylmethyl, 2-fluoro-4-trifluoromethylphenylmethanesulfonylmethyl, 2-fluoro-5-trifluoromethylphenylmethanesulfonylmethyl, 4-fluoro-3-trifluoromethylphenylmethanesulfonylmethyl, 2-methoxy-phenylmethanesulfonylmethyl, 3,5bis-trifluoromethyl-phenylmethanesulfonylmethyl, 4-difluoromethoxy-phenylmethanesulfonylmethyl, 2-difluoromethoxy-phenylmethanesulfonylmethyl, 3-difluoromethoxy-phenylmethanesulfonylmethyl, 2,6-dichloro-phenylmethanesulfonylmethyl, biphenyl-4-ylmethanesulfonylmethyl, 3,5-dimethyl-isoxazol-4-ylmethanesulfonylmethyl, 5-chloro-thiophen-2-ylmethanesulfonylmethyl, 2-[4-(1,1-Difluoro-methoxy)-benzenesulfonyl]-ethyl, 2-[2-(1,1-Difluoro-methoxy)-benzenesulfonyl]-ethyl, 2-[3-(1,1-Difluoro-methoxy)-benzenesulfonyl]-ethyl, 2-(4-trifluoromethoxy-benzenesulfonyl)-ethyl, 2-(3-trifluoromethoxy-benzenesulfonyl)-ethyl, 2-(2-trifluoromethoxy-benzenesulfonyl)-ethyl, (cyanomethyl-methyl-carbamoyl)-methyl, butyl, biphenyl-3-ylmethyl, 2-oxo-2-pyrrolidin-1-yl-ethyl, 2-benzenesulfonyl-ethyl, isobutylsulfanylmethyl, 2-phenylsulfanyl-ethyl, cyclohexylmethanesulfonylmethyl, 2-cyclohexyl-ethanesulfonyl, benzyl, naphthalen-2-yl, benzylsulfanylmethyl, 2-trifluoromethyl-benzylsulfanylmethyl, 5-bromo-thiophen-2-ylmethyl, phenylsulfanyl-ethyl and cyclopropylmethanesulfonylmethyl.

R⁴ more preferably is phenylamino, benzylamino, 4-phenoxy-phenylamino, phenethylamino, 3-phenyl-propylamino, morpholin-4-yl, cyclohexylamino, naphthalen-1-ylmethyl-amino, pyridin-3-ylamino, 6-methoxy-pyridin-3-ylamino, diisobutylamino, 4-nitro-benzylamino, 2-thiophen-2-yl-ethylamino, 3-phenoxy-phenylamino, cyanomethyl-amino, (pyridin-3-ylmethyl)-amino, 5,6,7,8-tetrahydro-naphthalen-1-ylamino, 2-pyridin-2-yl-ethylamino, 2,3-dihydro-indol-1-yl, 3,4-dihydro-1H-isoquinolin-2-yl, cyclohexylmethyl-amino, 2-methoxy-benzylamino, 1-phenyl-ethylamino, (pyridin-4-ylmethyl)-amino, benzyl-methyl-amino, 3-nitro-benzylamino, 4-methoxy-phenylamino, 3-carbamoyl-phenylamino, 4-carbamoyl-phenylamino, (tetrahydro-furan-2-ylmethyl)-amino, 3,4-dihydro-2H-quinolin-1-yl, dimethylamino, butylmethylamino, diisopropylamino, propylmethylamino, 1-(benzooxazole-2-carbonyl)-propylamino and isobutylmethylamino.

Reference to the preferred embodiments set forth above is meant to include all combinations of particular and preferred groups.

Particular compounds of the invention are selected from the compounds formed by joining the acyl carbon atom (C*) of one of the fragments (A1 to A37) shown in Table 1 to the methine carbon atom (*CH*) of one of the fragments (B1 to B88) shown in Table 2, and joining the methine carbon atom (*CH*) of one of the fragments (B1 to B88) shown in Table 2 to the acyl carbon atom (C*) of one of the fragments (C1 to C36) depicted in Table 3.

TABLE 1

| | | | | | |
|---|---|---|---|---|---|
| A1 | 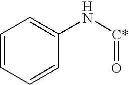 | A2 | 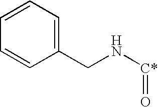 | A3 | 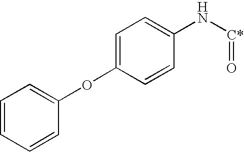 |
| A4 | 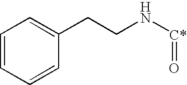 | A5 | 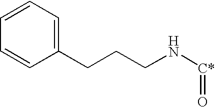 | A6 | 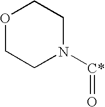 |
| A7 | 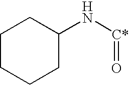 | A8 | 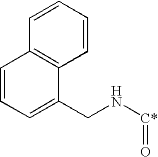 | A9 | 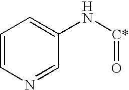 |
| A10 | 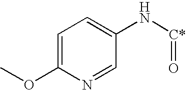 | A11 | 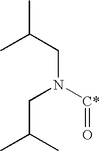 | A12 | 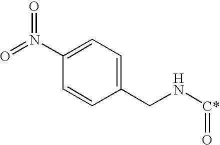 |
| A13 | 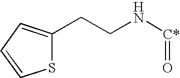 | A14 | 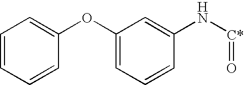 | A15 | 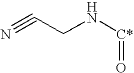 |
| A16 | 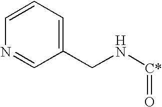 | A17 | 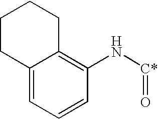 | A18 | 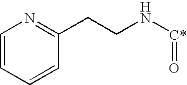 |
| A19 | 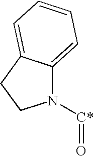 | A20 | 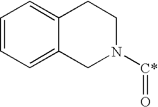 | A21 | 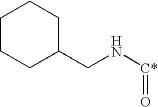 |
| A22 | 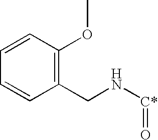 | A23 | 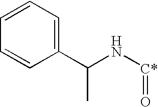 | A24 | 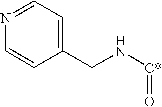 |
| A25 | 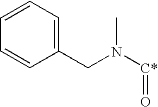 | A26 | 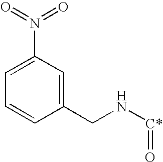 | A27 | 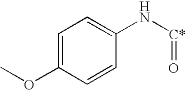 |
| A28 | 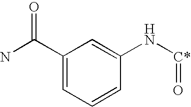 | A29 | 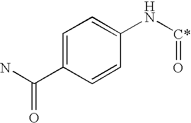 | A30 | 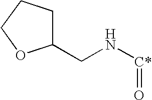 |
| A31 | 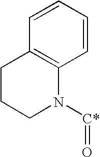 | A32 | 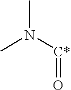 | A33 | 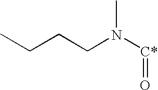 |
| A34 | 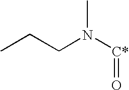 | A35 | 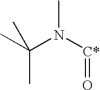 | A36 | 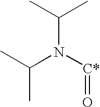 |
| A37 | 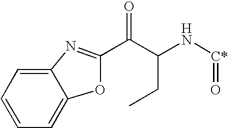 | | | | |

TABLE 2

| | | | | | |
|---|---|---|---|---|---|
| B1 | 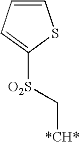 | B2 | 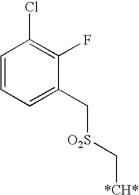 | B3 | 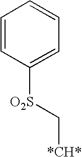 |
| B4 | 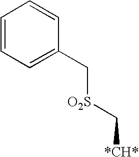 | B5 | 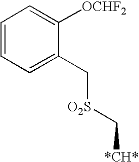 | B6 | 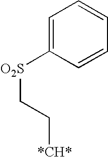 |
| B7 | 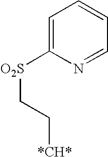 | B8 | 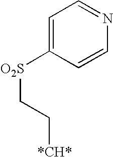 | B9 | 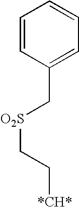 |
| B10 | 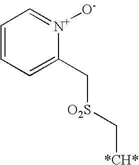 | B11 | 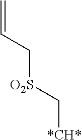 | B12 | 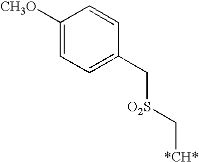 |
| B13 | 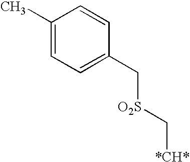 | B14 | 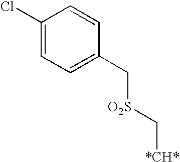 | B15 | 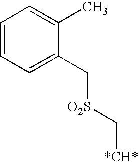 |
| B16 | 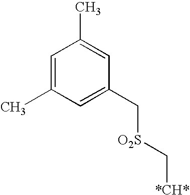 | B17 | 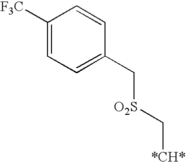 | B18 | 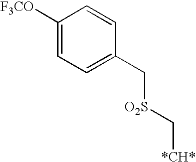 |
| B19 | 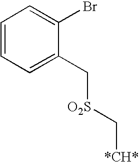 | B20 | 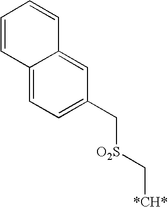 | B21 | 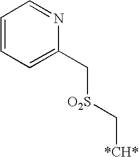 |
| B22 | 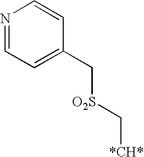 | B23 | 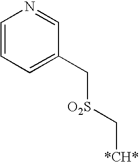 | B24 | 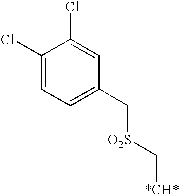 |
| B25 | 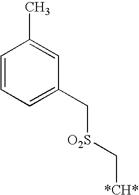 | B26 | 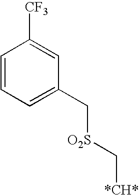 | B27 | 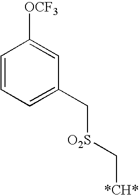 |
| B28 | 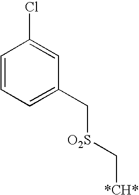 | B29 | 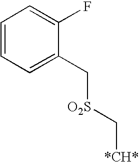 | B30 | 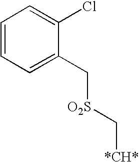 |
| B31 | 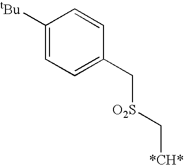 | B32 | 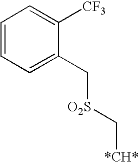 | B33 | 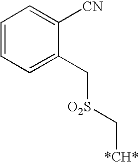 |
| B34 | 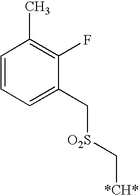 | B35 | 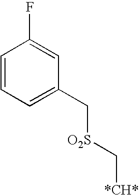 | B36 | 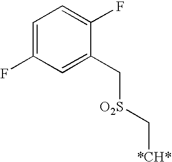 |
| B37 | 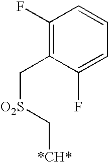 | B38 | 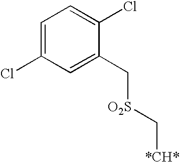 | B39 | 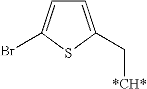 |
| B40 | 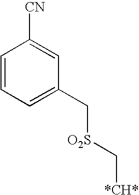 | B41 | 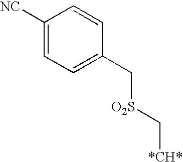 | B42 | 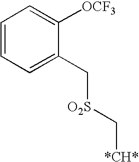 |
| B43 | 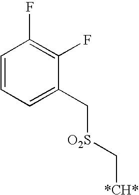 | B44 | 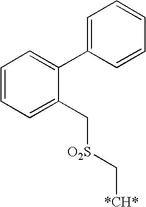 | B45 | 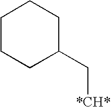 |
| B46 | 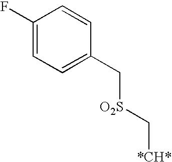 | B47 | 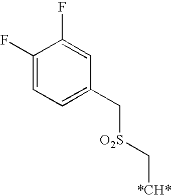 | B48 | 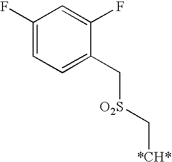 |
| B49 | 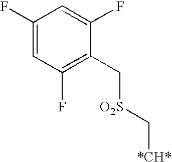 | B50 | 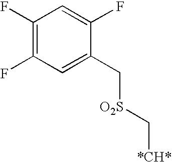 | B51 | 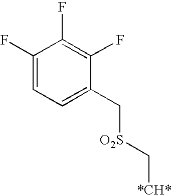 |
| B52 | 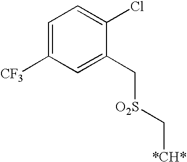 | B53 | 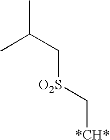 | B54 | 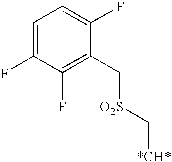 |
| B55 | 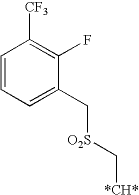 | B56 | 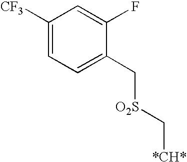 | B57 | 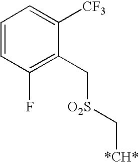 |
| B58 | 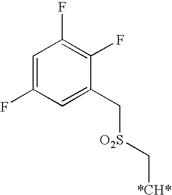 | B59 | 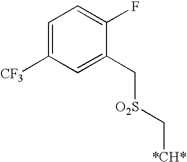 | B60 | 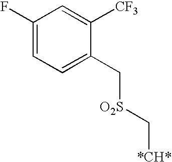 |
| B61 | 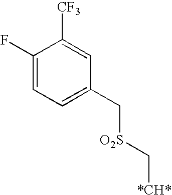 | B62 | 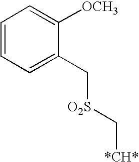 | B63 | 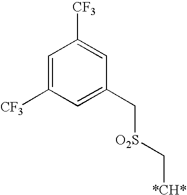 |
| B64 | 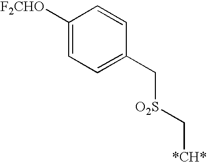 | B65 | 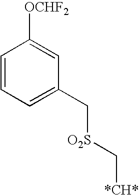 | B66 | 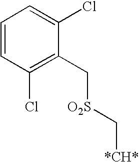 |
| B67 | 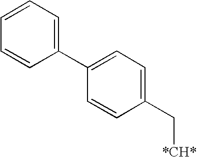 | B68 | 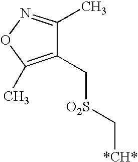 | B69 | 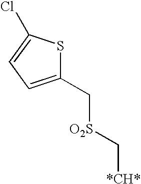 |
| B70 | 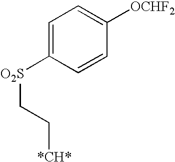 | B71 | 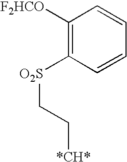 | B72 | 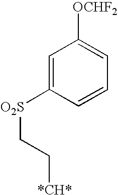 |
| B73 | 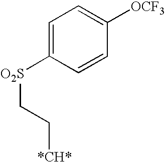 | B74 | 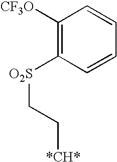 | B75 | 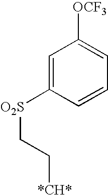 |
| B76 | 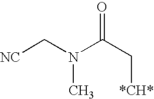 | B77 | 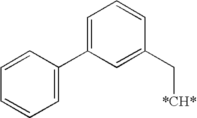 | B78 | 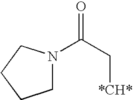 |
| B79 | 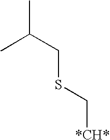 | B80 | 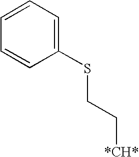 | B81 | 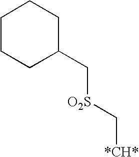 |
| B82 | 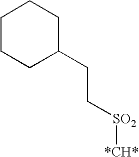 | B83 | 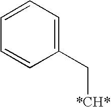 | B84 | 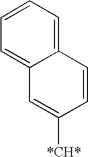 |
| B85 | 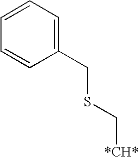 | B86 | 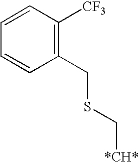 | B87 | 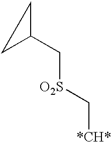 |
| B88 | 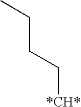 | | | | |

TABLE 3

| | | | | | |
|---|---|---|---|---|---|
| C1 | 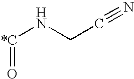 | C2 | 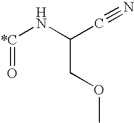 | C3 | 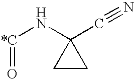 |
| C4 | 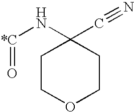 | C5 | 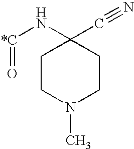 | C6 | 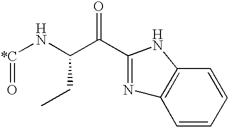 |
| C7 | 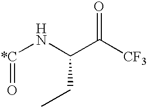 | C8 | 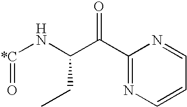 | C9 | 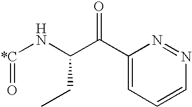 |
| C10 | 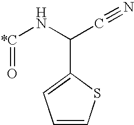 | C11 | 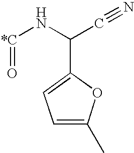 | C12 | 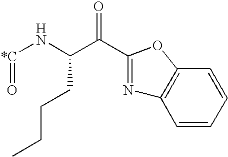 |
| C13 | 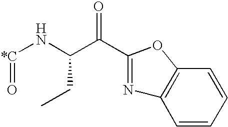 | C14 | 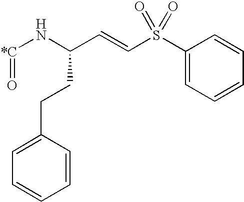 | C15 | 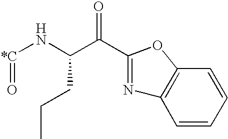 |
| C16 | 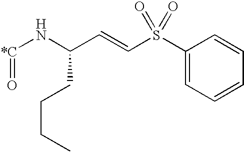 | C17 | 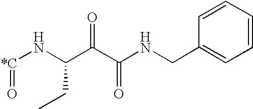 | C18 | 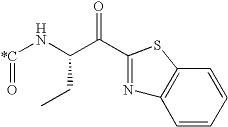 |
| C19 | 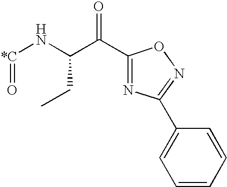 | C20 | 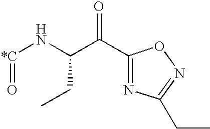 | C21 | 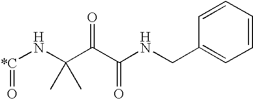 |
| C22 | 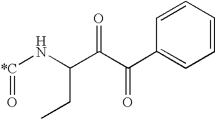 | C23 | 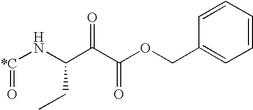 | C24 | 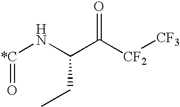 |
| C25 | 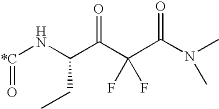 | C26 | 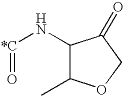 | C27 | 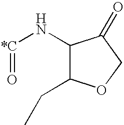 |
| C28 | 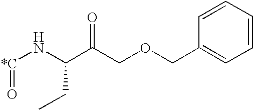 | C29 | 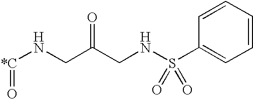 | C30 | 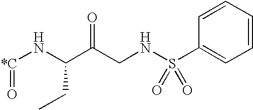 |
| C31 | 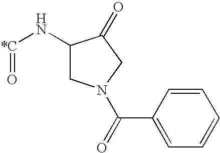 | C32 | 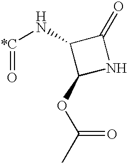 | C33 | 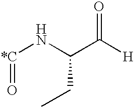 |
| C34 | 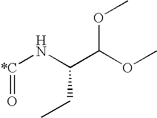 | C35 | 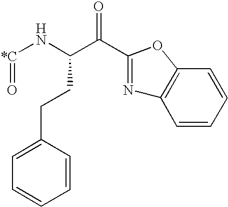 | C36 | 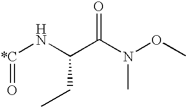 |

Particularly preferred compounds of “A”, “B”, and “C” combinations are illustrated in table 4, material submitted on compact disk along with this application, which is incorporated herein by reference. Two copies of the compact disk (copy 1 and copy 2) each contains a single 1.42-megabyte file named “table 4” both of which were created in ASCII format on May 31, 2001.

Thus, for example, in table 4 the compound denoted as A2-B45-C35 is the product of the combination of group A2 in Table 1 and B45 in Table 2 and C35 in Table 3, namely N-[1-(1-benzooxazol-2-yl-methanoyl)-3-phenyl-propyl]-N′-benzyl-2-cyclohexylmethyl-malonamide:

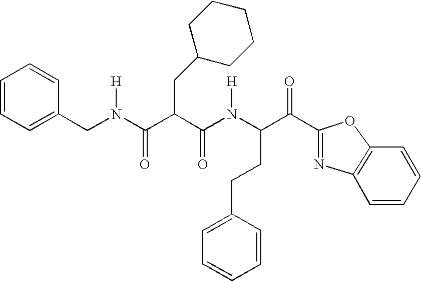

Further preferred are compounds of Formula I selected from a group consisting of:

2-butyl-N-cyanomethyl-N′-phenyl-malonamide (Compound 1; denoted as A1-B88-C1);

N-Cyanomethyl-2-cyclohexylmethyl-N′-phenyl-malonamide (Compound 2; denoted as A1-B45-C1);

N-Cyanomethyl-2-cyclohexylmethyl-N′-phenethyl-malonamide (Compound 3; denoted as A4-B45-C1);

N-Cyanomethyl-2-cyclohexylmethyl-N′-pyridin-4-ylmethyl-malonamide (Compound 4; denoted as A24-B45-C1);

N-[1-(Benzooxazole-2-carbonyl)-3-phenyl-propyl]-N′-benzyl-2-cyclohexylmethyl-malonamide (Compound 5; denoted as A2-B45-C35);

N-Cyanomethyl-N′-cyclohexyl-2-cyclohexylmethyl-malonamide (Compound 6; denoted as A7-B45-C1);

N-Benzyl-N′-cyanomethyl-2-cyclohexylmethyl-malonamide (Compound 7; denotes as A2-B45-C1);

N-Cyanomethyl-2-cyclohexylmethyl-N′-(4-phenoxy-phenyl)-malonamide (Compound 8; denoted as A3-B45-C1);

N-Cyanomethyl-2-cyclohexylmethyl-N′-(3-phenyl-propyl)-malonamide (Compound 9; denoted as A5-B45-C1);

N-Cyanomethyl-2-cyclohexylmethyl-3-morpholin-4-yl-3-oxo-propionamide (Compound 10; denoted as A6-B45-C1);

N-Cyanomethyl-2-cyclohexylmethyl-N′-naphthalen-1-ylmethyl-malonamide (Compound 11; denoted as A8-B45-C1);

N-Cyanomethyl-2-cyclohexylmethyl-N′-pyridin-3-yl-malonamide (Compound 12; denoted as A9-B45-C1);

N-Cyanomethyl-2-cyclohexylmethyl-N′,N′-diisobutyl-malonamide (Compound 13; denoted as A11-B45-C1);

N-Cyanomethyl-2-cyclohexylmethyl-N′,N′-diisopropyl-malonamide (Compound 14; denoted as A36-B45-C1);

N-Cyanomethyl-2-cyclohexylmethyl-N′-(6-methoxy-pyridin-3-yl)-malonamide (Compound 15; denoted as A10-B45-C1);

N-Cyanomethyl-2-cyclohexylmethyl-N′-(2-thiophen-2-yl-ethyl)-malonamide (Compound 16; denoted as A13-B45-C1);

N-Cyanomethyl-2-cyclohexylmethyl-N′-(3-phenoxy-phenyl)-malonamide (Compound 17; denoted as A14-B45-C1);

N-Cyanomethyl-2-cyclohexylmethyl-N′-(4-nitro-benzyl)-malonamide (Compound 18; denoted as A12-B45-C1);

N,N′-Bis-cyanomethyl-2-cyclohexylmethyl-malonamide (Compound 19; denoted as A15-B45-C1);

N-Cyanomethyl-2-cyclohexylmethyl-N′-(5,6,7,8-tetrahydro-naphthalen-1-yl)-malonamide (Compound 20; denoted as A17-B45-C1);

N-Cyanomethyl-2-cyclohexylmethyl-N′-(2-pyridin-2-yl-ethyl)-malonamide (Compound 21; denoted as A18-B45-C1);

N-Cyanomethyl-2-cyclohexylmethyl-3-(2,3-dihydro-indol-1-yl)-3-oxo-propionamide (Compound 22; denoted as A19-B45-C1);

N-Cyanomethyl-2-cyclohexylmethyl-3-(3,4-dihydro-1H-isoquinolin-2-yl)-3-oxo-propionamide (Compound 23; denoted as A20-B45-C1);

N-Cyanomethyl-2,N′-bis-cyclohexylmethyl-malonamide (Compound 24; denoted as A21-B45-C1);

N-Cyanomethyl-2-cyclohexylmethyl-N′-(2-methoxy-benzyl)-malonamide (Compound 25; denoted as A22-B45-C1);

N-Cyanomethyl-2-cyclohexylmethyl-N′-(1-phenyl-ethyl)-malonamide (Compound 26; denoted as A23-B45-C1);

N-Benzyl-N′-cyanomethyl-2-cyclohexylmethyl-N-methyl-malonamide (Compound 27; denoted as A25-B45-C1);

N-Cyanomethyl-2-cyclohexylmethyl-N′-(3-nitro-benzyl)-malonamide (Compound 28; denoted as A26-B45-C1);

N-Cyanomethyl-2-cyclohexylmethyl-N′-(4-methoxy-benzyl)-malonamide (Compound 29; denoted as A27-B45-C1);

N-(3-Carbamoyl-phenyl)-N′-cyanomethyl-2-cyclohexylmethyl-malonamide (Compound 30; denoted as A28-B45-C1);

N-Cyanomethyl-2-cyclohexylmethyl-N′-pyridin-3-ylmethyl-malonamide (Compound 31; denoted as A16-B45-C1);

N-(4-carbamoylphenyl)-N′-cyanomethyl-2-cyclohexylmethylmalonamide (Compound 32; denoted as A29-B45-C1);

N-cyanomethyl-2-cyclohexylmethyl-N′-tetrahydrofur-2-ylmethylmalonamide (Compound 33; denoted as A30-B45-C1);

N-cyanomethyl-2-cyclohexylmethyl-3-(3 4-dihydro-2H-quinolin-1-yl)-3-oxopropionamide (Compound 34; denoted as A3 1-B45-C1);

N-tert-butyl-N′-cyanomethyl-2-cyclohexylmethyl-N-methylmalonamide (Compound 35; denoted as A35-B45-C1);

N-cyanomethyl-2-cyclohexylmethyl-N′-methyl-N′-propylmalonamide (Compound 36; denoted as A34-B45-C1);

N-butyl-N′-cyanomethyl-2-cyclohexylmethyl-N-methylmalonamide (Compound 37; denoted as A33-B45-C1);

N-cyanomethyl-2-cyclohexylmethyl-N′,N′-dimethylmalonamide (Compound 38; denoted as A32-B45-C1);

N-benzyl-N′-cyanomethyl-2-(2-phenylsulfanylethyl)malonamide (Compound 39; denoted as A2-B80-C1);

2-(2-phenylsulfonylethyl)-N-benzyl-N′-cyanomethylmalonamide (Compound 40; denoted as A2-B6-C1);

2-(2-Benzenesulfonyl-ethyl)-N-[(S)-1-(1-benzooxazol-2-yl-methanoyl)-pentyl]-N′-benzyl-malonamide (Compound 41; denoted as A2-B6-C12);

N,N′-Bis-[(S)-1-(1-benzooxazol-2-yl-methanoyl)-propyl]-2-cyclohexylmethyl-malonamide (Compound 42; denoted as A37-B45-C13);

and the N-oxide derivatives, prodrug derivatives, protected derivatives, individual stereoisomers and mixtures of isomers thereof, and the pharmaceutically acceptable salts and solvates (e.g. hydrates) of such compounds and the N-oxide derivatives, prodrug derivatives, protected derivatives, individual isomers and mixtures of isomers thereof.

Pharmacology and Utility:

The compounds of the invention are selective inhibitors of cathepsin S and, as such, are useful for treating diseases in which cathepsin S activity contributes to the pathology and/or symptomatology of the disease. For example, the compounds of the invention are useful in treating autoimmune disorders, including, but not limited to, juvenile onset diabetes, multiple sclerosis, pemphigus vulgaris, Graves' disease, myasthenia gravis, systemic lupus erythemotasus, rheumatoid arthritis and Hashimoto's thyroiditis, allergic disorders, including, but not limited to, asthma, and allogeneic immune responses, including, but not limited to, organ transplants or tissue grafts.

Cathepsin S also is implicated in disorders involving excessive elastolysis, such as chronic obstructive pulmonary disease (e.g., emphysema), bronchiolitis, excessive airway elastolysis in asthma and bronchitis, pneumonities and cardiovascular disease such as plaque rupture and atheroma. Cathepsin S is implicated in fibril formation and, therefore, inhibitors of cathepsins S are of use in treatment of systemic amyloidosis.

The cysteine protease inhibitory activities of the compounds of the invention can be determined by methods known to those of ordinary skill in the art. Suitable in vitro assays for measuring protease activity and the inhibition thereof by test compounds are known. Typically, the assay measures protease induced hydrolysis of a peptide based substrate. Details of assays for measuring protease inhibitory activity are set forth in Examples 6-9, infra.

Administration and Pharmaceutical Compositions:

In general, compounds of Formula I will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula I may range from about 1 micrograms per kilogram body weight (μg/kg) per day to about 1 milligram per kilogram body weight (mg/kg) per day, typically from about 10 μg/kg/day to about 0.1 mg/kg/day. Therefore, a therapeutically effective amount for a 80 kg human patient may range from about 100 μg/day to about 100 mg/day, typically from about 1 μg/day to about 10 mg/day. In general, one of ordinary skill in the art, acting in reliance upon personal knowledge and the disclosure of this Application, will be able to ascertain a therapeutically effective amount of a compound of Formula I for treating a given disease.

The compounds of Formula I can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula I in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula I in the composition may vary widely depending upon the type of formulation, size of a unit dosage, kind of excipients and other factors known to those of skill in the art of pharmaceutical sciences. In general, a composition of a compound of Formula I for treating a given disease will comprise from 0.01% w to 1 0% w, preferably 0.30% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula I are described in Example 10.

Chemistry:

Processes for Making Compounds of Formula I:

Compounds of the invention may be prepared by the application or adaptation of known methods, by which is meant methods used heretofore or described in the literature, for example those described by R. C. Larock in Comprehensive Organic Transformations, VCH publishers, 1989.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

Compounds of Formula I, where X¹ is —NHC(R¹)(R²)X², can be prepared by proceeding as in the following Reaction Scheme 1: 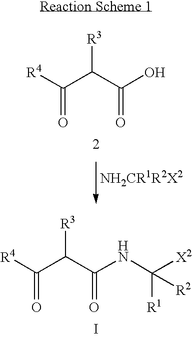
in which X², R¹, R², R³ and R⁴ are as defined in the Summary of the Invention.

Compounds of Formula I can be prepared by condensing an acid of Formula 2 with a compound of formula NH₂CR¹R²X². The condensation reaction can be effected with an appropriate coupling agent (e.g., benzotriazol-1-yloxytrispyrrolidino-phosphonium hexafluorophosphate (PyBOP®), 1-(3-dimethylaminopropyl)-3-ethylcarbodiimide hydrochloride (EDCI), O-benzotriazol-1-yl-N,N,N′,N′-tetramethyluronium hexafluorophosphate (HBTU), 1,3-dicyclohexyl-carbodiimide (DCC), or the like) and optionally an appropriate catalyst (e.g., 1-hydroxybenzotriazole (HOBt), 1-hydroxy-7-azabenzotriazole (HOAt), or the like) and non-nucleophillic base (e.g., N-methylmorpholine, triethylamine, or the like, or any suitable combination thereof) in a suitable solvent (N-methylpyrrolidinone, or the like) at ambient temperature and requires 3 to 10 hours to complete the reaction. A detailed description for the synthesis of a compound of Formula I by the processes in Reaction Scheme 1 is set forth in the Examples, infra.

Compounds of Formula I, where X¹ is —NHX³, can be prepared by proceeding as in the following Reaction Scheme 2: 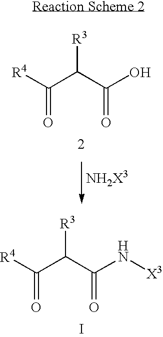
in which X³, R³ and R⁴ are as defined in the Summary of the Invention.

Compounds of Formula I can be prepared by condensing an acid of Formula 2 with a compound of formula NH₂X³. The condensation reaction can be effected with an appropriate coupling agent (e.g., benzotriazol-1-yloxytrispyrrolidino-phosphonium hexafluorophosphate (PyBOP®), 1-(3-dimethylaminopropyl)-3-ethylcarbodiimide hydrochloride (EDCI), O-benzotriazol-1-yl-N,N,N′,N′-tetramethyluronium hexafluorophosphate (HBTU), 1,3-dicyclohexyl-carbodiimide (DCC), or the like) and optionally an appropriate catalyst (e.g., 1-hydroxybenzotriazole (HOBt), 1-hydroxy-7-azabenzotriazole (HOAt), or the like) and non-nucleophillic base (e.g., N-methylmorpholine, triethylamine, or the like, or any suitable combination thereof) in a suitable solvent (N-methylpyrrolidinone, or the like) at ambient temperature and requires 3 to 10 hours to complete the reaction. A detailed description for the synthesis of a compound of Formula I by the processes in Reaction Scheme 1 is set forth in the Examples, infra.

Additional Processes for Preparing Compounds of Formula I:

A compound of Formula I can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula I can be prepared by reacting the free acid form of the compound with a pharmaceutically acceptable inorganic or organic base. Inorganic and organic acids and bases suitable for the preparation of the pharmaceutically acceptable salts of compounds of Formula I are set forth in the definitions section of this Application. Alternatively, the salt forms of the compounds of Formula I can be prepared using salts of the starting materials or intermediates.

The free acid or free base forms of the compounds of Formula I can be prepared from the corresponding base addition salt or acid addition salt form. For example, a compound of Formula I in an acid addition salt form can be converted to the corresponding free base by treating with a suitable base (e.g., ammonium hydroxide solution, sodium hydroxide, and the like). A compound of Formula I in a base addition salt form can be converted to the corresponding free acid by treating with a suitable acid (e.g., hydrochloric acid, etc).

The N-oxides of compounds of Formula I can be prepared by methods known to those of ordinary skill in the art. For example, N-oxides can be prepared by treating an unoxidized form of the compound of Formula I with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of Formula I can be prepared from the N-oxide of an appropriate starting material.

Compounds of Formula I in unoxidized form can be prepared from N-oxides of compounds of Formula I by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds of Formula I can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound of Formula I with a suitable carbamylating agent (e.g., 1,1-acyloxyalkylcarbonochloridate, para-nitrophenyl carbonate, or the like).

Protected derivatives of the compounds of Formula I can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999. Compounds of the present invention may be conveniently prepared, or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol. Compounds of Formula I can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of Formula I, dissociable complexes are preferred (e.g., crystalline diastereoisomeric salts). Diastereomers have distinct physical properties (e.g., melting points, boiling points, solubilities, reactivity, etc.) and can be readily separated by taking advantage ofthese dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

In summary, the compounds of Formula I are made by a process which comprises:
(A) reacting a compound of Formula 2:

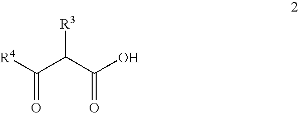

with a compound of formula NH₂CR¹R²X², in which R¹, R², R³, R⁴ and X² are as defined in the Summary of the Invention for Formula I; or
(B) reacting a compound of Formula 2 with a compound of Formula NH₂X³, in which R³, R⁴ and X³ are as described in the Summary of the Invention for Formula I; and
(C) optionally converting a compound of Formula I into a pharmaceutically acceptable salt;
(D) optionally converting a salt form of a compound of Formula I to non-salt form;
(E) optionally converting an unoxidized form of a compound of Formula I into a pharmaceutically acceptable N-oxide;
(F) optionally converting an N-oxide form of a compound of Formula I its unoxidized form;
(G) optionally resolving an individual isomer of a compound of Formula I from a mixture of isomers;
(H) optionally converting a non-derivatized compound of Formula I into a pharmaceutically prodrug derivative; and
(I) optionally converting a prodrug derivative of a compound of Formula I to its non-derivatized form.

### EXAMPLES

The present invention is further exemplified, but not limited by, the following examples that illustrate the preparation of compounds of Formula I (Examples) and intermediates (References) according to the invention.

### Reference 1

### 2-Phenylcarbamoyl-hexanoic acid

A solution of aniline (5.47 ml, 60 mmol) and triethylamine (8.36 ml, 60 mmol) in methylene chloride (150 ml) was cooled to −20° C. and treated with methylmalonylchloride (8.36 ml, 60 mmol) in methylene chloride (20 ml). The reaction mixture was allowed to warm to ambient temperature for 3 hours and then poured into cold IN HCl. The organic layer was separated and washed with aqueous sodium bicarbonate then brine and dried over magnesium sulfate and evaporated to give methyl 2-phenylcarbamoylacetate.

A mixture of methyl 2-phenylcarbamoylacetate (1.159 g, 6 mmol) lithium hydroxide (0.43 g, 18 mmol) and 1-iodobutane (0.91 ml, 8 mmol) in N-methylpyrrolidinone (10 ml) was stirred at ambient temperature for 1.5 hours. The reaction mixture was poured into ice water, extracted with ethylacetate (twice, 50 ml each). The combined extracts were washed with brine, dried over magnesium sulfate and evaporated. The residue was purified by flash chromatography on silica gel eluting with 20% ethylacetate/hexane to give methyl 2-phenylcarbamoylhexanoate (0.715 g, 48% yield).

A solution of methyl 2-phenylcarbamoylhexanoate (0.98 g 3.9 mmol) in methanol (10 ml) was treated with sodium hydroxide (4 ml, 4 mmol) at ambient temperature for 17 hours. The methanol was removed under reduced pressure and the residue was treated with IN HCl and extracted with ethylacetate (twice, 50 ml each). The organic layers were washed with brine, dried over magnesium sulfate and evaporated to give 2-phenylcarbamoylhexanoic acid (0.68 g, 2.9 mmol, 74% yield).

### Reference 2

### 2-Cyclohexylmethyl-N-phenyl-malonamic acid

A mixture of methyl 2-phenylcarbamoylacetate (prepared as in reference Example 1) (4.39 g, 22.7 mmol), lithium hydroxide (1.08 g, 45 mmol) and bromomethylcyclohexane (3.76 ml, 27 mmol), in N-methylpyrrolidinone (25 ml) was stirred at ambient temperature for 17 hours. The reaction mixture was poured into ice water and extracted with ether (three times, 100 ml each). The extracts were washed with water then brine, dried over magnesium sulfate and evaporated. The residue was purified by flash chromatography on silica gel eluting with 10% ethylacetate (hexane) to give methyl 2-cyclohexylmethyl-N-phenyl malonamate (1.89 g, 6.5 mmol, 29% yield). The aqueous layer above was cooled on ice and acidified to pH 2 with IN HCl. The aqueous layer was extracted with ether (3 times, 100 ml each) and the extracts were washed with water, then brine, dried over magnesium sulfate and evaporated to give 2-cyclohexylmethyl-N-phenyl malonamic acid (1.12 g, 18% yield).

### Reference 3

### 2-Cyclohexylmethyl-N-phenethyl-malonamic acid

Sodium (6.9 g, 0.3 mol), dissolved in ethanol (300 ml), and then diethylmalonate (50.3 ml, 0.3 mol) was added. Bromomethylcyclohexane (46 ml, 0.33 mol) was added and the reaction mixture was heated at 70° C. for 14 hours. The reaction mixture was cooled and the ethanol removed by evaporation. The resulting mass was dissolved in ice water and then extracted with ethylacetate. The organic layers were washed with water, then brine and dried over magnesium sulfate. The solvents were removed under reduced pressure to give diethylcyclohexyl malonate.

A solution of diethylcyclohexylmalonate (12.8 g, 0.05 mol) in ethanol (100 ml) was treated with a solution of lithium hydroxide (1.2 g, 0.05 moles) in water (50 ml) and then stirred at ambient temperature for 15 hours. The ethanol was removed at reduced pressure and water (50 ml) was added to the residue. The reaction mixture was extracted with ether, cooled on ice and acidified to pH 1.5 with HCl. The aqueous phase was saturated with NaCl and extracted with ethylacetate (twice, 150 ml each). Drying over magnesium sulfate and evaporating the solvent gave ethyl 2-cyclohexylmalonate (8.52 g, 37 mmol, 74% yield).

The ethyl 2-cyclohexylmalonate (8.52 g, 37 mmol) in ethylacetate (80 ml) was cooled to 0° C. and treated with dimethylformade (50 μL) and then oxalylchloride (3.93 ml, 45 mmol). The reaction temperature was raised to room temperature and after 2 hours the solvents were removed under reduced pressure to give ethyl 2-cyclohexylmalonyl chloride.

The malonylchloride above was diluted to 28 ml volume with ethylacetate and 2 ml of that solution was added to a solution of phenethylamine (0.376 ml, 3 mmol) and N-methylmorpholine (0.40 g, 4 mmol) in ethylacetate (4 ml) at −20° C. After 15 minutes the reaction mixture was allowed to warm to ambient temperature overnight. The reaction mixture was diluted with ethylacetate (5 ml) and ice water (5 ml). The organic layer was separated and washed with cold 0.05 N HCl, then aqueous NaHCO₃, then brine, dried over magnesium sulfate and evaporated under reduced pressure. The residue was purified by radial chromatography to give ethyl 2-cyclohexylmethyl-N-phenethyl malonamate (0.366 g, 1.10 mmol, 42% yield).

The ester above (0.366 g, 1.10 mmol) in ethanol (10 ml) was treated at ambient temperature with aqueous sodium hydroxide (1.3 ml of IN) for 2.5 hours. The reaction mixture was diluted with water (30 ml) and washed with ether (3 times, 30 ml each). The aqueous layer was cooled, acidified with IN HCl (2 ml) and extracted with ethylacetate (3 times, 30 ml each). The ethylacetate extracts were washed with brine, dried over magnesium sulfate and evaporated to give 2-cyclohexylmethyl-N-phemethyl malonamic acid (0.138 g, 0.46 mmol, 42% yield).

### Reference 4

### 2-Cyclohexylmethyl-N-pyridin-4-ylmethyl-malonamic acid

Ethyl 2-cyclohexylmethylmalonyl chloride, prepared as in Reference 3 (0.307 g, 1.25 mmol), was condensed with 4-aminomethyl pyridine using the method of Reference 3 to give ethyl 2-cyclohexylmethyl-4-pyrin-4-ylmethylmalonamate (0.237 g, 0.74 mmol, 58% yield).

This ester was hydrolyzed with sodium hydroxide using the method of Reference 3 to give 2-cyclohexylmethyl-N-pyridin-4-ylmethylmalonamic acid (0.041 g, 0.14 mmol, 19% yield).

Proceeding as in the above referenced examples provided the following compounds:

N-Benzyl-2-cyclohexylmethyl-malonamic acid;

2-Cyclohexylmethyl-N-(4-phenoxy-phenyl)-malonamic acid;

2-Cyclohexylmethyl-N-(3-phenyl-propyl)-malonamic acid;

2-Cyclohexylmethyl-3-morpholin-4-yl-3-oxo-propionic acid;

N-Cyclohexyl-2-cyclohexylmethyl-malonamic acid;

2-Cyclohexylmethyl-N-naphthalen-1-ylmethyl-malonamic acid;

2-Cyclohexylmethyl-N-pyridin-3-yl-malonamic acid;

2-Cyclohexylmethyl-N,N-diisobutyl-malonamic acid;

2-Cyclohexylmethyl-N-(6-methoxy-pyridin-3-yl)-malonamic acid;

2-Cyclohexylmethyl-N-(2-thiophen-2-yl-ethyl)-malonamic acid;

2-Cyclohexylmethyl-N-(3-phenoxy-phenyl)-malonamic acid;

2-Cyclohexylmethyl-N-(4-nitro-benzyl)-malonamic acid;

N-Cyanomethyl-2-cyclohexylmethyl-malonamic acid;

2-Cyclohexylmethyl-N-(5,6,7,8-tetrahydro-naphthalen-1-yl)-malonamic acid;

2-Cyclohexylmethyl-N-(2-pyridin-2-yl-ethyl)-malonamic acid;

2-Cyclohexylmethyl-3-(2,3-dihydro-indol-1-yl)-3-oxo-propionic acid;

2-Cyclohexylmethyl-3-(3,4-dihydro-1H-isoquinolin-2-yl)-3-oxo-propionic acid;

2,N-Bis-cyclohexylmethyl-3-oxo-butyramide;

2-Cyclohexylmethyl-N-(2-methoxy-benzyl)-3-oxo-butyramide;

2-Cyclohexylmethyl-N-(1-phenyl-ethyl)-malonamic acid;

N-Benzyl-2-cyclohexylmethyl-N-methyl-malonamic acid;

2-Cyclohexylmethyl-N-(3-nitro-benzyl)-3-oxo-butyramide;

2-Cyclohexylmethyl-N-(4-methoxy-benzyl)-malonamic acid;

N-(3-Carbamoyl-phenyl)-2-cyclohexylmethyl-malonamic acid;

2-Cyclohexylmethyl-N-pyridin-3-ylmethyl-malonamic acid;

N-(4-Carbamoyl-phenyl)-2-cyclohexylmethyl-malonamic acid;

2-Cyclohexylmethyl-N-(tetrahydro-furan-2-ylmethyl)-malonamic acid;

2-Cyclohexylmethyl-3-(3,4-dihydro-2H-quinolin-1-yl)-3-oxo-propionic acid;

N-tert-Butyl-2-cyclohexylmethyl-N-methyl-malonamic acid;

2-Cyclohexylmethyl-N-methyl-N-propyl-malonamic acid;

N-Butyl-2-cyclohexylmethyl-N-methyl-malonamic acid;

2-Cyclohexylmethyl-N,N-dimethyl-malonamic acid;

(R)-2-Benzylcarbamoyl-4-phenylsulfanyl-butyric acid; and

4-Benzenesulfonyl-2-benzylcarbamoyl-butyric acid;

### Example 1

### 2-butyl-N-cyanomethyl-N′-phenylmalonamide

### Compound 1

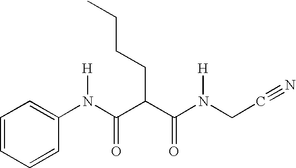

A solution comprised of 2-Phenylcarbamoyl-hexanoic acid (188 g, 0.8 mmol), prepared as in Reference 1, in DMF (5.0 mL) was treated with PyBOP® (425 0.8 mmol), aminoacetonitrile bisulfate (140 mg, 0.9 mmol) and triethylamine (600 μL, 4.3 mmol). The mixture was stirred for 3 hours and then partitioned between water (20 mL) and ethyl acetate (50 mL). The organic layer was separated and washed with 1 M saturated sodium bicarbonate solution, 1 M hydrochloric acid solution and water, dried (MgSO₄) and concentrated. Product was purified from the residue by flash column on silica gel (60° A) with 50% ethyl acetate in hexane to provide 2-butyl-N-cyanomethyl-N′-phenylmalonamide (125 mg, 57% yield). ¹H NMR: (DMSO) 10.01 (s, 1H), 7.59 (d, J=8 Hz, 2H), 7.31 (t, J=7 Hz, 2H), 7.06 (t, J=7 Hz, 1H), 4.13 (d, J=6 Hz, 2H), 3.32 (t, J=8 Hz, 1H), 1.80 (m, 2H), 1.25 (m, 4H), 0.86 (t, J=7 Hz, 3H). MS: m/e 273.9.

### Example 2

### N-Cyanomethyl-2-cyclohexylmethyl-N′-phenyl-malonamide

### Compound 2

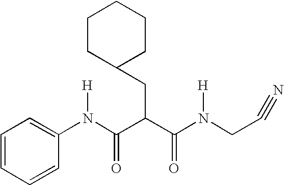

A solution comprised of 2-Cyclohexylmethyl-N-phenyl-malonamic acid (350 mg, 1.2 mmol), prepared as in Reference 2, EDCI (250 mg, 1.3 mmol), HOBt hydrate (199 mg, 1.3 mmol), amino acetonitrilebisulfate (200 mg, 1.3 mmol) and N-methylmorpholine (0.30 ml, 2.7 mmol) in N,N-dimethylpyrrolidinone (5 ml) was stirred at ambient temperature for 15 hours. The reaction mixture was poured into cold IN HCl and extracted with ethylacetate. The organic phase was washed with aqueous saturated sodium bicarbonate and then brine (50 ml each) dried over magnesium sulfate and evaporated. The residue was purified by radial chromatography using 50% ethylacetate/hexane as eluent to provide N-Cyanomethyl-2-cyclohexylmethyl-N′-phenyl-malonamide (179 mg, 48% yield). ¹H NMR: (DMSO) 10.01 (s, 1H), 8.47 (t, J=5 Hz, 1H), 7.59 (d, J=7 Hz, 2H), 7.31 (t, J=8 Hz, 2H), 7.07 (t, J=7 Hz, 1H), 4.13 (d, J=5 Hz, 2H), 3.47 (t, J=7 Hz, 1H), 1.6 (m, 7H), 1.1 (m, 4H), 0.9 (m, 2H). MS: m/e 313.2.

### Example 3

### N-cyanomethyl-2-cyclohexylmethyl-N′-phenethylmalonamide

### Compound 3

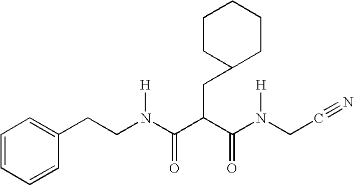

A solution comprised of 2-Cyclohexylmethyl-N-phenethyl-malonic acid (138 mg, 0.46 mmol), prepared as in Reference 3, EDCI (115 mg, 0.60 mmol), HOBt hydrate (92 mg, 0.60 mmol), N-methylmorpholine (0115 ml, 1.38 mmol) in N,N-dimethyl pyrrolidimone (4 ml) was stirred at ambient temperature for 10 min. Aminoacetonitrile bisulfate (106 mg, 0.69 mmol) was added. The reaction mixture was stirred at ambient temperature for 2 hours, then poured into cold IN HCl and extracted twice with ethylacetate (50 ml each). The organic phase was washed with aqueous sodium bicarbonate and then brine (50 ml each), dried over magnesium sulfate and evaporated. The residue was purified by radial chromatography using 50% ethylacetate/hexane as eluent to provide N-cyanomethyl-2-cyclohexylomethyl-N′-phenethylmalonamide (56 mg, 36% yield). ¹H NMR: (DMSO) 8.38 (t, J=5 Hz, 1H), 7.98 (t, J=6 Hz, 1H), 7.25 (m, 5H), 4.10 (d, J=6 Hz, 2H), 3.2 (m, 3H), 2.71 (t, J=7 Hz, 2H), 1.6 (m, 7H), 1.1 (m, 4H), 0.85 (m, 2H). MS: m/e 342.10.

### Example 4

### N-cyanomethyl-2-cyclohexylmethyl-N′-pyrid-4-ylmethylmalonamide

### Compound 4

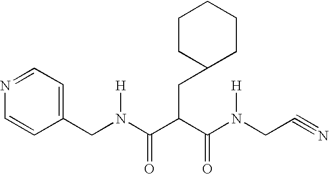

A solution comprised of 2-Cyclohexylmethyl-N-pyridin-4-ylmethyl-malonic acid (41 mg, 0.14 mmol), prepared as in Reference 4, was coupled to aminoacetonitrile as described in Example 3 to provide N-cyanomethyl-2-cyclohexylmethyl-N′-pyrid-4-ylmethylmalonamide (13 mg, 28% yield). ¹HNMR: (DMSO) 8.5 (m,4H),7.20 (d, J=6 Hz, 2H), 4.3 (m, 2H), 4.13 (d, J=5 Hz, 2H), 3.3 (m, 1H), 1.6 (m, 7H), 1.1 (m, 4H), 0.83 (m, 2H). MS: m/e 329.08.

### Example 5

### N-[1-(1-Benzooxazol-2-yl-methanoyl)-3-phenyl-propyl]-N′-benzyl-2-cyclohexylmethylmalonamide

### Compound 5

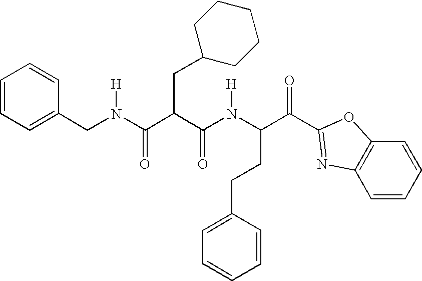

A mixture of N-benzyl-2-cyclohexylmethyl-malonamic acid (200 mg, 0.69 mmol), HOBt (159 mg, 1.04 mmol), EDC (146 mg, 0.76 mmol), 2-amino-1-benzooxazol-2-yl-4-phenyl-butan-1-one (195 mg, 0.69 mmol), dichloromethane (3 mL) and triethylamine (106 μL, 0.76 mmol) was allowed to stir 2 hour. The product was extracted into ethyl acetate (60 mL) and washed with two 15 mL portions of 1N HCl, and two 15 mL portions of saturated NaHSO₃, dried over MgSO₄ and concentrated. Ethyl acetate (5 mL) was added and a white precipitate formed and was collected to give N-[1-(1-benzooxazol-2-yl-1-hydroxy-methyl)-3-phenyl-propyl]-N′-benzyl-2-cyclohexylmethyl-malonamide (81 mg, 0.12 mmol, 21% yield).

N-[1-(1-Benzooxazol-2-yl-1-hydroxy-methyl)-3-phenyl-propyl]-N′-benzyl-2-cyclohexylmethyl-malonamide (70 mg, 0.126 mmol) was dissolved in 0.6 mL dichloromethane and treated with Dess Martin periodinane (107 mg, 0.253 mmol). The mixture was stirred for 2 hours, then 8 mL of 0.26M NaS₂O₃ in saturated NaHSO₃ was added and the mixture was extracted with two 15 mL portions of ethyl acetate and washed with two 4 mL portions of saturated NaHSO₃. The organic layer was dried over MgSO₄ and concentrated. The product was recrystallized from ethyl acetate and hexane to give N-[1-(1-Benzooxazol-2-yl-methanoyl)-3-phenyl-propyl]-N′-benzyl-2-cyclohexylmethyl-malonamide (40 mg, 0.072 mmol, 57% yield); ¹H NMR: (DMSO) 7.88 (m, 1H), 7.68-7.40 (m, 3H), 7.35-7.10 (m, 10H), 6.90 (m, 1H), 5.65 (m, 1H), 4.43 (d, J=5.7 Hz, 2H), 3.25 (m, 1H), 2.74 (t, J=8.0 Hz, 1H), 2.46 (m, 1H), 2.17 (m, 1H), 1.77 (t, J=7.4 Hz, 1H), 1.64 (m, 7H), 1.22 (m, 4H), 0.87 (m, 2H); MS: (M⁺+1) 552.8; 551.68.

The following compounds of Formula I were provided by proceeding as in the above Examples:

N-cyanomethyl-N′-cyclohexyl-2-cyclohexylmethylmalonamide (Compound 6); ¹H NMR(DMSO): 8.31 (t, J=6 Hz, 1H), 7.82 (d, J=8 Hz, 1H), 4.10 (d, J=8 Hz, 2H), 3.52 (m, 1H), 3.20 (t, J=7 Hz, 1H), 1.6 (m, 12H), 1.1 (m, 9H), 0.83 (m, 2H); MS (m/e)=320.11;

N-benzyl-N′-cyanomethyl-2-cyclohexylmethylmalonamide (Compound 7); ¹H NMR(DMSO): 8.45 (m, 2H), 7.3 (m, 5H), 4.33 (dd, J=6.15 Hz, 1H), 4.23 (dd, J=6.15 Hz, 1H), 4.12 (d, 2H), 3.3 (m, 1H), 1.6 (m, 7H), 1. 1 (m, 4H), 0.85 (m, 2H); MS (m/e)=328.15, M.Wt.=327.43;

N-cyanomethyl-2-cyclohexylmethyl-N′-(4-phenoxyphenyl)malonamide (Compound 8); ¹H NMR(DMSO): 10.1 (s, 1H), 8.50 (t, J=5 Hz, 1H), 7.61 (d, J=7 Hz, 2H), 7.37 (t, J=7 Hz, 2H), 7.11 (t, J=7 Hz, 1H), 7.0(m,4H), 4.14(d, J=5 Hz, 2H), 3.47 (t, J=7 Hz, 1H), 1.7 (m, 7H), 1.1 (m, 4H), 0.92 (m, 2H); MS (m/e)=406.10, M.Wt.=405.49;

N-cyanomethyl-2-cyclohexylmethyl-N′-(3-phenylpropyl)malonamide (Compound 9); ¹H NMR(DMSO): 8.38 (t, J=6 Hz, 1H), 8.00 (t, J=6 Hz, 1H), 7.2 (m, 5H), 4.10 (d, J=5 Hz, 2H), 3.23 (t, J=7 Hz, 1H), 3.1 (m, 2H), 2.5 (m, 2H), 1.6 (m, 9H), 1.1 (m, 4H), 0.85 (m, 2H); MS (m/e)=356.02;

N-cyanomethyl-2-cyclohexylmethyl-3-morpholin-4-yl-3-oxopropionamide (Compound 10); ¹HNMR(DMSO): 8.54 (t, J=4 Hz, 1H), 4.12 (d, J=5 Hz, 2H), 3.5 (m, 8H), 1.65 (m, 8H), 1.15 (m, 4H), 0.85 (m, 2H); MS (m/e)=308.05;

N-cyanomethyl-2-cyclohexylmethyl-N′-naphth-1-ylmethylmalonamide (Compound 11); ¹H NMR(DMSO): 8.53 (t, J=5 Hz, 1H), 8.43 (t, J=6 Hz, 1H), 8.04 (m, 1H), 7.94 (m, 1H), 7.86 (d, J=8 Hz, 1H), 7.5 (m, 4H), 4.85 (dd, J=6.15 Hz, 1H), 4.65 (dd, J=5.15 Hz, 1H), 4.12 (d, J=3 Hz, 2H), 3.3 (m, 1H), 1.6 (m, 8H), 1.0 (m, 5H); MS (m/e)=378.18, M.Wt. 377.18;

N-cyanomethyl-2-cyclohexylmethyl-N′-pyrid-3-ylmalonamide (Compound 12); ¹H NMR(DMSO): 10.24 (s, 1H), 8.75 (s, 1H), 8.54 (t, J=5 Hz, 1H), 8.29 (d, J=5 Hz, 1H), 8.04 (d, J=7 Hz, 1H), 7.36 (m, 1H), 4.13 (d, J=5 Hz, 2H), 3.49 (t, J=7 Hz, 1H), 1.7 (m, 7H), 1.1 (m, 4H), 0.9 (m, 2H); MS (m/e)=314.91;

N-cyanomethyl-2-cyclohexylmethyl-N′,N′-diisobutylmalonamide (Compound 13); ¹H NMR(DMSO): 8.50 (t, J=4 Hz, 1H), 4.09 (m, 2H), 3.63 (t, J=7 Hz, 1H), 3.2 (m, 2H), 3.05 (m, 2H), 1.9 (m, 2H), 1.6 (m, 7H), 1.1 (m, 4H), 0.8 (m, 14H); MS (m/e)=350.08, M.Wt. 349.51;

N-cyanomethyl-2-cyclohexylmethyl-N′,N′-diisopropylmalonamide (Compound 14); ¹HNMR(DMSO): 8.45 (t, J=5 Hz, 1H), 4.1 (m, 3H), 3.55 (t, J=7 Hz, 1H), 3.46 (m, 1H), 1.6 (m, 7H), 1.27 (d, J=7 Hz, 6H), 1.11 (m, 10H), 0.85 (m, 2H); MS (m/e)=321.99, M.Wt. 321.24;

N-cyanomethyl-2-cyclohexylmethyl-N′-(6-methoxyrid-3-yl)malonamide (Compound 15); ¹HNMR(DMSO): 10.04 (s, 1H), 8.50 (t, J=5 Hz, 1H), 8.35 (s, 1H), 7.88 (d, J=9 Hz, 1H), 6.80 (d, J=9 Hz, 1H), 4.13 (m, 2H), 3.81 (s, 3H), 3.44 (t, J=8 Hz, 1H), 1.7 (m, 7H), 1.1 (m, 4H), 0.91 (m, 2H); MS (m/e)=345.01, M.Wt. 344.18;

N-cyanomethyl-2-cyclohexylmethyl-N′-(2-thien-2-ylethyl)malonamide (Compound 16); ¹HNMR(DMSO): 8.40 (t, J=5 Hz, 1H), 8.07 (t, J=5 Hz, 1H), 7.33 (d, J=5 Hz, 1H), 6.95 (m, 1H), 6.87 (m, 1H), 4.10 (d, J=5 Hz, 2H), 3.3 (m, 3H), 3.21 (t, J=7 Hz, 2H), 1.6 (m, 7H), 1.1 (m, 4H), 0.85 (m, 2H); MS (m/e)=348.09, M.Wt. 347.48;

N-cyanomethyl-2-cyclohexylmethyl-N′-(3-phenoxyphenyl)malonamide (Compound 17); ¹H NMR(DMSO): 10.1 (s, 1H), 8.45 (t, J=5 Hz, 1H), 7.41 (t, J=8 Hz, 2H), 7.33 (m, 3H), 7.16 (t, J=7 Hz, 1H), 7.03 (d, J=8 Hz, 2H), 6.73 (m, 1H), 4.1 (m, 2H), 3.42 (t, J=7 Hz, 1H), 1.6 (m, 7H), 1.1 (m, 4H), 0.85 (m, 2H); MS (m/e)=406.04, M.Wt. 405.49;

N-cyanomethyl-2-cyclohexylmethyl-N′-(4-nitrobenzyl)malonamide (Compound 18); ¹HNMR(DMSO): 8.61 (t, J=6 Hz, 1H), 8.53 (t, J=6 Hz, 1H), 8.17 (d, J=9 Hz, 2H), 7.47 (d, J=9 Hz, 2H), 4.41 (d, J=6 Hz, 2H), 4.14 (d, J=6 Hz, 2H), 3.3 (m, 1H), 1.6 (m, 7H), 1.1 (m, 4H), 0.87 (m, 2H); MS (m/e)=373.02, M.Wt. 372.42;

N,N′-biscyanomethyl-2-cyclohexylmethylmalonamide (Compound 19); ¹H NMR(DMSO): 8.59 (t, J=5 Hz, 2H), 4.14 (d, J=6 Hz, 4H), 3.28 (t, J=8 Hz, 1H), 1.6 (m, 7H), 1.1 (m, 4H), 0.86 (m, 2H); MS (m/e)=276.99, M.Wt. 276.34;

N-cyanomethyl-2-cyclohexylmethyl-N′-(5,6,7,8-tetrahydronaphth-1-yl)malonamide (Compound 20); ¹HNMR(DMSO): 9.28 (s, 1H), 8.57 (t, J=6 Hz, 1H),7.21 (d, J=8 Hz, 1H), 7.05 (t, J=8 Hz, 1H), 6.90 (d, J=7 Hz, 1H), 4.16 (d, J=6 Hz, 2H), 3.49 (t, J=7 Hz, 1H), 2.7 (m, 2H), 2.5 (m, 2H), 1.7 (m, 1H), 1.1 (m, 4H), 0.91 (m, 2H); MS (m/e)=368.04, M.Wt. 367.48;

N-cyanomethyl-2-cyclohexylmethyl-N′-(2-pyrid-2-ylethyl)malonamide (Compound 21); ¹H NMR(DMSO): 8.49 (m, 1H), 8.40 (t, J=6 Hz, 1H), 8.00 (t, J=5 Hz, 1H), 7.68 (dt, J=2.8 Hz, 1H), 7.2 (m, 2H), 4.09 (d, J=6 Hz, 2H), 3.42 (m, 2H), 3.17 (t, J=8 Hz, 1H), 2.85 (t, J=7 Hz, 2H), 1.6 (m, 7H), 1.07 (m, 4H), 0.83 (m, 2H); MS (m/e)=343.04, M.Wt. 342.44;

N-cyanomethyl-2-cyclohexylmethyl-3-(2,3-dihydroindol-1-yl)-3-oxopropionamide (Compound 22); ¹H NMR(DMSO): 8.73 (t, J=5 Hz, 1H), 8.06 (d, J=8 Hz, 1H), 7.24 (d, J=7 Hz, 1H), 7.15 (t, J=7 Hz, 1H), 7.00 (t, J=7 Hz, 1H), 4.15 (d, J=5 Hz, 2H), 4.1 (m, 2H), 3.66 (t, J=7 Hz, 1H), 3.14 (m, 2H), 1.6 (m, 7H), 1.1 (m, 4H), 0.91 (m, 2H); MS (m/e)=340.07, M.Wt. 339.19;

N-cyanomethyl-2-cyclohexylmethyl-3-(3,4-dihydro-1H-isoquinolin-2-yl)-3-oxopropionamide (Compound 23); ¹H NMR(DMSO): 8.64(t, J=5 Hz, 1H), 7.15 (s, 4H), 4.65 (m, 2H), 4.11 (t, J=6 Hz, 2H), 3.77 (m, 2H), 2.8 (m, 2H), 1.7 (m, 7H), 1.1 (m, 4H), 0.89 (m, 2H), 3.54 (m, 1H); MS (m/e)=354.05, M.Wt. 353.46;

N-cyanomethyl-2,N′-biscyclohexylmethylmalonamide (Compound 24); ¹H NMR(DMSO): 8.34 (t, J=5 Hz, 1H), 7.93 (t, J=5 Hz, 1H), 4.09 (d, J=6 Hz, 2H), 3.23 (dd, J=7.9 Hz, 1H), 3.0 (m, 1H), 2.8 (m, 1H), 1.6 (m, 12H), 1.4 (m, 1H), 1.1 (m, 7H), 0.86 (m, 4H); MS (m/e)=334.00, M.Wt. 333.47;

N-cyanomethyl-2-cyclohexylmethyl-N′-(2-methoxybenzyl)malonamide (Compound 25); ¹H NMR(DMSO): 8.44 (t, J=5 Hz, 1H), 8.27 (t, J=6 Hz, 1H), 7.24 (dt, J=2.7 Hz, 1H), 7.10 (dd, J=2.7 Hz, 1H), 6.96 (d, J=7 Hz, 1H), 6.88 (dt, J=7.1 Hz, 1H), 4.30 (dd, J=6.16 Hz, 1H), 4.20 (dd, J=5.16 Hz, 1H), 4.12 (d, J=6 Hz, 2H), 3.79 (s, 3H), 3.3 (m, 1H), 1.6 (m, 7H), 1.l (m, 4H), 0.85 (m, 2H); MS (m/e)=358.03, M.Wt. 357.45;

N-cyanomethyl-2-cyclohexylmethyl-N′-(1-phenylethyl)malonamide (Compound 26); ¹HNMR(DMSO): 8.25 (m, 1H), 7.4 (m, 5H), 4.02 (m, 2H), 3.18 (s, 3H), 3.25 (m, 1H), 1.6 (m, 7H), 1.1 (m, 4H), 0.7 (m, 2H); MS (m/e)=328.08, M.Wt. 327.42;

N-benzyl-N′-cyanomethyl-2-cyclohexylmethyl-N-methylmalonamide (Compound 27); ¹HNMR(DMSO): 8.62 (m, 1H), 7.3 (m, 5H), 4.5 (m, 2H), 4.13 (d, J=6 Hz, 2H), 3.7 (m, 1H), 2.93 (s, 3H), 1.6 (m, 7H), 1.1 (m, 4H), 0.88 (m, 2H); MS (m/e)=342.09, M.Wt. 341.45;

N-cyanomethyl-2-cyclohexylmethyl-N′-(3-nitrobenzyl)malonamide (Compound 28); ¹HNMR(DMSO): 8.6 (t, 1H), 8.5 (t, 1H), 8.1 (m, 2H), 7.6 (m, 2H), 4.1 (m, 2H), 1.6 (m, 7H), 1.1 (m, 4H), 0.8 (m, 2H); MS (m/e)=373.07, M.Wt. 372.42;

N-cyanomethyl-2-cyclohexylmethyl-N′-(4-methoxybenzyl)malonamide (Compound 29); ¹HNMR(DMSO): 8.42 (t, J=5 Hz, 1H), 8.38 (t, J=6 Hz, 1H), 7.14 (d, J=9 Hz, 2H), 6.86 (d, J=9 Hz, 2H), 4.25 (dd, J=6.15 Hz, 1H), 4.15 (dd, J=7.16 Hz, 1H), 3.71 (s, 3H), 3.27 (t, J=8 Hz, 1H), 1.6 (m, 7H), 1.1 (m, 4H), 0.84 (m, 2H), 4.11 (d, J=6 Hz, 2H); MS (m/e)=356.97, M.Wt. 357.45;

N-(3-carbamoylphenyl)-N′-cyanomethyl-2-cyclohexylmethylmalonamide (Compound 30); ¹HNMR(DMSO): 10.14 (s, 1H), 8.48 (t, 1H), 8.03 (s, 1H), 7.75 (d, 1H), 7.54 (d, 1H), 7.35 (m, 2H), 4.12 (d, 2H), 3.4 (t, 1H), 1.6 (m, 7H), 1.1 (m, 4H), 0.9 (m, 2H), 3.29 (s, 3H); MS (m/e)=357.11, M.Wt. 356.42;

N-cyanomethyl-2-cyclohexylmethyl-N′-pyrid-3-ylmethylmalonamide (Compound 31); ¹HNMR(DMSO): 8.4 (m, 4H), 7.55 (d, 1H), 7.25 (dd, 1H), 4.28 (dd, 1H), 4.18 (dd, 1H), 4.05 (d, 2H), 3.2 (m, 1H), 1.6 (m, 7H), 1.01 (m, 4H), 0.78 (m, 2H); MS (m/e)=329.03, M.Wt. 328.41;

N-(4-carbamoylphenyl)-N′-cyanomethyl-2-cyclohexylmethylmalonamide (Compound 32); ¹H NMR(DMSO): 10.22 (s, 2H), 8.50 (t, J=6 Hz, 1H), 7.83 (d, J=9 Hz 2H), 7.64 (d, J=9 Hz, 2H), 7.52 (s, 1H), 4.13 (d, J=6 Hz, 2H), 3.48 (t, J=7 Hz, 1H), 1.7 (m, 7H), 1.1 (m, 4H), 0.9 (m, 2H); MS (m/e)=357.04, M.Wt. 356.42;

N-cyanomethyl-2-cyclohexylmethyl-N′-tetrahydrofur-2-ylmethylmalonamide (Compound 33); ¹H NMR(DMSO): 8.38 (t, J=5 Hz, 1H), 7.98 (t, J=4 Hz, 1H), 4.10 (d, J=6 Hz 2H), 3.8 (m, 2H), 3.6 (m, 1H), 3.2 (m, 4H), 1.8 (m, 3H), 1.6 (m, 7H), 1.1 (m, 4H), 0.85 (m, 2H); MS (m/e)=322.02, M.Wt. 321.41;

N-cyanomethyl-2-cyclohexylmethyl-3-(3 4-dihydro-2H-quinolin-1-yl)-3-oxopropionamide (Compound 34); ¹H NMR(DMSO): 8.5 (m, 1H), 7.35 (m, 1H), 7.2 (m, 4H), 4.1 (m, 2H), 3.82 (dd, 1H), 2.78 (t, 1H), 2.72 (t, 1H), 2.59 (m, 1H), 1.8 (m, 2H), 1.5 (m, 7H), 1.0 (m, 4H), 0.7 (m, 2H); MS (m/e)=354.02, M.Wt. 353.46;

N-tert-butyl-N′-cyanomethyl-2-cyclohexylmethyl-N-methylmalonamide (Compound 35); ¹H NMR(DMSO): 8.41 (t, J=5 Hz, 1H), 4.09 (d, J=5 Hz, 1H), 3.56 (t, J=7 Hz, 1H), 2.86 (s, 3H), 1.6 (m, 7H), 1.31 (s, 9H), 1.1 (m, 4H), 0.8 (m, 2H); MS (m/e)=308.04, M.Wt. 307.43;

N-cyanomethyl-2-cyclohexylmethyl-N′-methyl-N′-propylmalonamide (Compound 36); ¹HNMR(DMSO): 8.5 (m, 1H), 4.10 (m, 2H), 3.60 (t, J=7 Hz, 1H), 3.2 (m, 2H), 2.96 (s, 3H), 1.65 (m, 7H), 1.45 (m, 2H), 1.1 (m, 4H), 0.8 (m, 5H); MS (m/e)=294.02, M.Wt. 293.40;

N-butyl-N′-cyanomethyl-2-cyclohexylmethyl-N-methylmalonamide (Compound 37); ¹HNMR(DMSO): 8.5 (m, 1H), 4.10 (d, J=5 Hz, 2H), 3.60 (t, 1H), 3.3 (m, 2H), 2.95 (s, 3H), 1.6 (m, 7H), 1.4 (m, 2H), 1.1 (m, 6H), 0.8 (m, 5H); MS (m/e)=308.01, M.Wt. 307.43;

N-cyanomethyl-2-cyclohexylmethyl-N′,N′-dimethylmalonamide (Compound 38); ¹H NMR(DMSO): 8.55 (t, J=5 Hz, 1H), 4.11 (d, J=7 Hz, 2H), 3.62 (t, J=8 Hz, 1H), 2.99 (s, 3H), 2.81 (s, 3H), 1.6 (m, 7H), 1.1 (m, 4H), 0.85 (m, 2H); MS (m/e)=266.01, M.Wt. 265.18;

N-benzyl-N′-cyanomethyl-2-(2-phenylsulfanylethyl)malonamide (Compound 39); ¹HNMR(DMSO): 8.56 (t, J=6 Hz, 1H), 8.49 (t, J=6 Hz, 1H), 7.3 (m, 10H), 4.29 (d, J=6 Hz, 2H), 4.14 (d, J=6 Hz, 2H), 3.40 (t, J=7 Hz, 1H), 2.86 (t, J=8 Hz, 2H), 2.05 (m, 2H); MS (m/e)=368.02, M.Wt. 367.14; and

2-(2-phenylsulfonylethyl)-N-benzyl-N′-cyanomethylmalonamide (Compound 40); ¹H NMR(DMSO): 8.56 (t, J=6 Hz, 1H), 8.43 (t, J=6 Hz, 1H), 7.86 (d, J=7 Hz, 2H), 7.79 (t, J=5 Hz, 1H), 7.68 (t, J=8 Hz, 2H), 7.25 (m, 5H), 4.26 (d, J=6 Hz, 2H), 4.13 (d, J=6 Hz, 2H), 3.36 (m, 1H), 3.19 (m, 2H), 2.00 (m, 2H); MS (m/e)=400.04, M.Wt. 399.47;

2-(2-Benzenesulfonyl-ethyl)-N-[(S)-1-(1-benzooxazol-2-yl-methanoyl)-pentyl]-N′-benzyl-malonamide (Compound 41) ¹HNMR(DMSO): 8.56 (d, J=6 Hz, 1H), 8.2 (m, 1H), 8.0-7.5 (m, 9H), 7.3-7.1 (m, 5H), 5.3 (m, 1H), 4.24 (t, J=6 Hz, 2H), 3.41 (t, J=7 Hz, 1H), 3.18(m,2H), 1.96(m,3H), 1.67(m, 1H), 1.30(m,4H),0.82(m, 3H); MS (m/e)=576.27, M.Wt. 575.21; and

N,N′-Bis-[(S)-1-(1-benzooxazol-2-yl-methanoyl)-propyl]-2-cyclohexylmethyl-malonamide (Compound 42) ¹H NMR(DMSO): 8.41 (d, J=6 Hz, 2H), 8.00 (d, J=8 Hz, 2H), 7.89 (d, J=8 Hz, 2H), 7.65 (t, J=7 Hz, 2H), 7.53 (t, J=8 Hz, 2H), 5.19 (m, 2H), 3.42 (t, J=8 Hz, 1H), 1.98 (m, 2H), 1.74 (m, 2H), 1.52 (m, 7H), 0.94 (m, 10H), 0.77 (m, 2H); MS (m/e)=572.26, M.Wt. 573.4.

### Example 6

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM). Human cathepsin S (0.158 pMoles in 25 μL of assay buffer) was added to the dilutions. The assay solutions were mixed for 5-10 seconds on a shaker plate, covered and incubated for 30 minutes at ambient temperature. Z-Val-Val-Arg-AMC (9 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

### Example 7

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at ambient temperature. Z-FR-AMC (20 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

### Example 8

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 minutes at ambient temperature. Z-Phe-Arg-AMC (4 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

### Example 9

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 minutes at ambient temperature. Z-Phe-Arg-AMC (1 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested according to the above-described assays for protease inhibition and observed to exhibit selective cathepsin S inhibitory activity. For example, the compounds of the invention were found to inhibit cathepsin S protease activity at concentrations that are least 50 fold less than those concentrations required to produce an equiactive inhibition of cathepsin K protease activity. The apparent inhibition constants (Kᵢ) for compounds of the invention, against Cathepsin S, were in the range from about 10⁻¹⁰M to about 10⁻⁷M.

### Example 10

Representative Pharmaceutical Formulations Containing a Compound of Formula I

ORAL FORMULATION

| | |
|---|---|
| Compound of Formula I | 10-100 mg |
| Citric Acid Monohydrate | 105 mg |
| Sodium Hydroxide | 18 mg |
| Flavoring | |
| Water | q.s. to 100 mL |

INTRAVENOUS FORMULATION

| | |
|---|---|
| Compound of Formula I | 0.1-10 mg |
| Dextrose Monohydrate | q.s. to make isotonic |
| Citric Acid Monohydrate | 1.05 mg |
| Sodium Hydroxide | 0.18 mg |
| Water for Injection | q.s. to 1.0 mL |

TABLET FORMULATION

| | |
|---|---|
| Compound of Formula I | 1% |
| Microcrystalline Cellulose | 73% |
| Stearic Acid | 25% |
| Colloidal Silica | 1%. |

We claim:

1. A compound of Formula I: 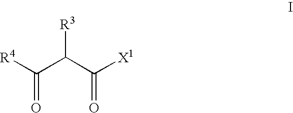
in which:
X¹ is —NHC(R¹)(R²)X² or —NHX³;
X² is cyano, —C(R⁷)(R⁸)X³, —C(R⁷)(R⁸)CF₃, —C(R⁷)(R⁸)CF₂CF₂R⁹—CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is (C₁₋₄)alkyl, (C₅₋₁₀)aryl(C₀₋₆)alkyl or (C₅₋₁₀)heteroaryl(C₀₋₆)alkyl; R⁶ is hydrogen or (C₁₋₆)alkyl; R⁷ is hydrogen or (C₁₋₄)alkyl and R⁸ is hydroxy or R⁷ and R⁸ together form oxo; R⁹ is hydrogen, halo, (C₁₋₄)alkyl, (C₅₋₁₀)aryl(C₀₋₆)alkyl or (C₅₋₁₀)heteroaryl(C₀₋₆)alkyl;
X³ comprises a heteromonocyclic ring containing 4 to 6 ring member atoms or a fused heterobicyclic ring system containing 8 to 14 ring member atoms and any carbocyclic ketone, iminoketone or thioketone derivative thereof;
wherein within R⁵, X² or X³ any alicyclic or aromatic ring system may be substituted further by 1 to 5 radicals independently selected from (C₁₋₆)alkyl, (C₁₋₆)alkylidene, cyano, halo, halo-substituted(C₁₋₄)alkyl, nitro, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴ S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³ and —X⁴S(O)₂R¹³ and/or 1 radical selected from —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴OC(O)R¹⁴, —X⁴NR¹⁴R¹², —X⁴NR¹²C(O)R¹⁴, —X⁴NR¹²C(O)OR¹⁴, —X⁴C(O)NR¹²R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹², wherein X⁴ is a bond or (C₁₋₆)alkyl; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen, halo or (C₁₋₆)alkyl and R² is selected from a group consisting of hydrogen, cyano, halo, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³, —X⁴S(O)₂R¹³, —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴OC(O)R¹⁴, —X⁴NR¹⁴R¹², —X⁴NR¹²C(O)R¹⁴, —X⁴NR¹²C(O)OR¹⁴, —XC(O)NR¹²R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹², wherein X⁴, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is optionally substituted with 1 to 3 radicals independently selected from (C₁₋₆)alkyl, (C₁₋₆)alkylidene, cyano, halo, halo-substituted(C₁₋₄)alkyl, nitro, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³, —X⁴S(O)₂R¹³ and —X⁴C(O)R¹³, wherein X⁴, R¹² and R¹³ are as defined above;
R³ is —C(R⁶)(R⁶)X⁵, wherein R⁶ is as defined above and X⁵ is selected from —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴C(O)R¹³, —X⁴NR¹²C(O)R¹³, —X⁴S(O)R¹³ and —X⁴S(O)₂R¹³, —R¹⁴, —X⁴OR¹⁴, —X⁴SR¹⁴, —X⁴S(O)R¹⁴, —X⁴S(O)₂R¹⁴, —X⁴C(O)R¹⁴, —X⁴C(O)OR¹⁴, —X⁴OC(O)R¹⁴, —X⁴NR¹⁴R¹², —X⁴NR¹²C(O)R¹⁴, —X⁴NR¹²C(O)OR¹⁴, —X⁴C(O)NR¹⁴R¹², —X⁴S(O)₂NR¹⁴R¹², —X⁴NR¹²S(O)₂R¹⁴, —X⁴NR¹²C(O)NR¹⁴R¹² and —X⁴NR¹²C(NR¹²)NR¹⁴R¹² wherein X⁴, R¹², R¹³ and R¹⁴ are as defined above;
R⁴ is —NR⁶R⁶, —NR⁶R¹⁴, —NR⁶R¹⁵ or —NR⁶X⁵C(O)R¹⁴ wherein R⁶, X⁵ and R¹⁴ are as described above and R¹⁵ is hydrogen, —(C₁₋₆)alkyl or —X⁵OR⁶ wherein X⁵ is as described above; or R⁶ and R¹⁵ together with the nitrogen atom to which R⁶ and R¹⁵ are attached form hetero(C₃₋₁₀)cycloalkyl, hetero(C₅₋₁₀)aryl or hetero(C₈₋₁₀)bicycloaryl;
wherein within R³ and R⁴ any alicyclic or aromatic ring system may be substituted further by 1-5 radicals independently selected from (C₁₋₆)alkyl, (C₁₋₆)alkylidene, cyano, halo, halo-substituted(C₁₋₄)alkyl, nitro, —X⁴NR¹²R¹², —X⁴NR¹²C(O)R¹², —X⁴NR¹²C(O)OR¹², —X⁴NR¹²C(O)NR¹²R¹², —X⁴NR¹²C(NR¹²)NR¹²R¹², —X⁴OR¹², —X⁴SR¹², —X⁴C(O)OR¹², —X⁴C(O)R¹², —X⁴OC(O)R¹², —X⁴C(O)NR¹²R¹², —X⁴S(O)₂NR¹²R¹², —X⁴NR¹²S(O)₂R¹², —X⁴P(O)(OR¹²)OR¹², —X⁴OP(O)(OR¹²)OR¹², —X⁴NR¹²C(O)R¹³, —X⁴S(O)R¹³, —X⁴C(O)R¹³ and —X⁴S(O)₂R¹³ and/or 1 radical selected from —R¹⁴, —X⁴OR¹⁴, —X⁴SR¹⁴, —X⁴S(O)R¹⁴, —X⁴S(O)₂R¹⁴, —X⁴ C(O)R¹⁴, —X⁴C(O)OR¹⁴, —X⁴OC(O)R¹⁴, —X⁴NR¹⁴R¹², —X⁴NR¹²C(O)R¹⁴, —X⁴NR¹²C(O)OR¹⁴, —X⁴C(O)NR¹⁴R¹², —X⁴S(O)₂NR¹⁴R¹², —X⁴NR S(O)₂R¹⁴, —X⁴NR¹²C(O)NR¹⁴R¹² and —X⁴NR¹²C(NR¹²)NR¹⁴R¹²; and within R³ and R⁴ any aliphatic moiety may be substituted further by 1-5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹²,—OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁴, R¹², R¹³ and R¹⁴ are as described above;
with the proviso that only one bicyclic ring structure is present within R³ or R⁴; and the N-oxide derivatives, prodrug derivatives, protected derivatives, individual isomers and mixtures of isomers thereof, and the pharmaceutically acceptable salts and solvates of such compounds and the N-oxide derivatives, prodrug derivatives, protected derivatives, individual isomers and mixtures of isomers thereof.

2. The compound of claim 1 in which:
X¹ is —NHC(R¹)(R²)X² or —NHX³;
X² is cyano, —C(O)X³, —C(O)CF₃, —C(O)CF₂CF₂R⁹, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ and R⁶ are as described above;
X³ comprises a heteromonocyclic ring containing 4 to 6 ring member atoms or a fused heterobicyclic ring system containing 8 to 14 ring member atoms and any carbocyclic ketone, iminoketone or thioketone derivative thereof;
wherein within R⁵, X² or X³ any alicyclic or aromatic ring system may be substituted further by 1 to 5 radicals independently selected from (C₁₋₆)alkyl or —X⁴OC(O)R¹² and/or 1 radical selected from —R¹⁴, —X⁴C(O)R¹⁴ or —X⁴OC(O)R¹⁴;
wherein X⁴, R¹² and R¹⁴ are as described above;
R¹ is hydrogen or (C₁₋₆)alkyl and R² is hydrogen, —X⁴OR¹², (C₅₋₁₀)heteroaryl(C₀₋₆)alkyl, (C₅₋₁₀)aryl(C₀₋₆)alkyl, (C₅₋₁₀)cycloalkyl(C₀₋₆)alkyl, (C₅₋₁₀)heterocycloalkyl(C₀₋₆)alkyl or (C₁₋₆)alkyl; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is optionally substituted with 1 to 3 radicals independently selected from (C₁₋₆)alkyl and hydroxy;
R³ is —CH₂X⁵, wherein X⁵ at each occurrence independently is selected from —X⁴SR¹², —X⁴C(O)NR¹²R¹², —X⁴S(O)₂R¹³, —X⁴C(O)R¹³, —X⁴SR¹⁴, —R¹⁴, —X⁴S(O)₂R¹⁴, —X⁴R¹², —X⁴C(O)R¹⁴, —X⁴C(O)NR¹⁴R¹², wherein X⁴, R¹², R¹³ and R¹⁴ are as defined above;
R⁴ is —NR⁶R⁶, —NR⁶R¹⁴, —NR⁶R¹⁵ or —NR⁶X⁵C(O)R¹⁴ wherein R⁶, X⁵ and R¹⁴ are as described above and R¹⁵ is hydrogen, —(C₁₋₆)alkyl or —X⁵OR⁶ wherein X⁵ is as described above; or R⁶ and R¹⁵ together with the nitrogen atom to which R⁶ and R¹⁵ are attached form hetero(C₃₋₁₀)cycloalkyl, hetero(C₅₋₁₀)aryl or hetero(C₈₋₁₀)bicycloaryl;
wherein within R³ and R⁴ any alicyclic or aromatic ring system may be substituted further by 1-5 radicals independently selected from (C₁₋₆)alkyl, cyano, halo, nitro, halo-substituted(C₁₋₄)alkyl, —X⁴OR¹², —X⁴C(O)OR¹², —C(O)R¹³, —X⁴C(O)NR¹²R¹², —X⁴NR¹²S(O)₂R¹² and/or 1 radical selected from —R¹⁴, —X⁴OR¹⁴ and —X⁴C(O)NR¹⁴R¹²; within R³ and R⁴ any aliphatic moiety may be substituted further by 1-5 radicals independently selected from cyano; wherein X⁴ , R¹², R¹³ and R¹⁴ are as described above; with the proviso that only one bicyclic ring structure is present within R³ or R⁴; and the N-oxide derivatives, prodrug derivatives, protected derivatives, individual isomers and mixtures of isomers thereof, and the pharmaceutically acceptable salts and solvates of such compounds and the N-oxide derivatives, prodrug derivatives, protected derivatives, individual isomers and mixtures of isomers thereof.

3. The compound of claim 2 in which:
X¹ is —NHC(R¹)(R²)X² or —NHX³;
X² is cyano, —C(O)X³, —CF₃, —CF₂CF₃, (E)-2-benzenesulfonyl-vinyl, 2-dimethylcarbamoyl-2,2-difluoro-acetyl, 1-benzylcarbamoyl-methanoyl, 1-benzyloxy(oxalyl), 2-benzyloxy-acetyl, 2-benzenesulfonylamino-ethanoyl or 2-oxo-2-phenyl-ethanoyl;
X³ is 1H-benzoimidazol-2-yl, pyrimidin-2-yl, benzooxazol-2-yl, benzothiazol-2-yl, pyridazin-3-yl, 3-phenyl-[1,2,4]oxadiazol-5-yl, 3-ethyl-[1,2,4]oxadiazol-5-yl, 2-methyl-4-oxo-tetrahydro-furan-3-yl, 2-ethyl-4-oxo-tetrahydro-furan-3-yl, 4-oxo-1-(1-phenyl-methanoyl)-pyrrolidin-3-yl or (S)-2-Acetoxy-4-oxo-azetidin-3-yl;
R¹ is hydrogen or methyl and R² is hydrogen, methoxymethyl, (C₁₋₆)alkyl, phenethyl, thiophen-2-yl or 5-methyl-furan-2-yl, or (ii) R¹ and R² taken together with the carbon atom to which both R¹ and R² are attached form cyclopropylene, tetrahydro-pyran-4-ylene or methyl-piperidin-4-ylene.

4. The compound of claim 3 in which R³ is selected from thiophene-2-sulfonylmethyl, 3-chloro-2-fluoro-phenylmethanesulfonylmethyl, benzenesulfonylmethyl, phenylmethanesulfonylmethyl, 2-(1,1-difluoro-methoxy)-phenylmethanesulfonylmethyl, 2-benzenesulfonyl-ethyl, 2-(pyridine-2-sulfonyl)-ethyl, 2-(pyridine-4-sulfonyl)-ethyl, 2-phenylmethanesulfonyl-ethyl, oxy-pyridin-2-ylmethanesulfonylmethyl, prop-2-ene-1-sulfonylmethyl, 4-methoxy-phenylmethanesulfonylmethyl, p-tolylmethanesulfonylmethyl, 4-chloro-phenylmethanesulfonylmethyl, o-tolylmethanesulfonylmethyl, 3,5-dimethyl-phenylmethanesulfonylmethyl, 4-trifluoromethyl-phenylmethanesulfonylmethyl, 4-trifluoromethoxy-phenylmethanesulfonylmethyl, 2-bromo-phenylmethanesulfonylmethyl, pyridin-2-ylmethanesulfonylmethyl, pyridin-3-ylmethanesulfonylmethyl, pyridin-4-ylmethanesulfonylmethyl, naphthalen-2-ylmethanesulfonylmethyl, 3-methyl-phenylmethanesulfonylmethyl, 3-trifluoromethyl-phenylmethanesulfonylmethyl, 3-trifluoromethoxy-phenylmethanesulfonylmethyl, 4-fluoro-2-trifluoromethoxy-phenylmethanesulfonylmethyl, 2-fluoro-6-trifluoromethyl-phenylmethanesulfonylmethyl, 3-chloro-phenylmethanesulfonylmethyl, 2-fluoro-phenylmethanesulfonylmethyl, 2-trifluoro-phenylmethanesulfonylmethyl, 2-cyano-phenylmethanesulfonylmethyl, 4-tert-butyl-phenylmethanesulfonylmethyl, 2-fluoro-3-methyl-phenylmethanesulfonylmethyl, 3-fluoro-phenylmethanesulfonylmethyl, 4-fluoro-phenylmethanesulfonylmethyl, 2-chloro-phenylmethanesulfonylmethyl, 2,5-difluoro-phenylmethanesulfonylmethyl, 2,6-difluoro-phenylmethanesulfonylmethyl, 2,5-dichloro-phenylmethanesulfonylmethyl, 3,4-dichloro-phenylmethanesulfonylmethyl, 2-(1,1-difluoro-methoxy)-phenylmethanesulfonylmethyl, 2-cyano-phenylmethanesulfonylmethyl, 3-cyano-phenylmethanesulfonylmethyl, 2-trifluoromethoxy-phenylmethanesulfonylmethyl, 2,3-difluoro-phenylmethanesulfonylmethyl, 2,5-difluoro-phenylmethanesulfonylmethyl, biphenyl-2-ylmethanesulfonylmethyl, cyclohexylmethyl, 3-fluoro-phenylmethanesulfonylmethyl, 3,4-difluoro-phenylmethanesulfonylmethyl, 2,4-difluoro-phenylmethanesulfonylmethyl, 2,4,6-trifluoro-phenylmethanesulfonylmethyl, 2,4,5-trifluoro-phenylmethanesulfonylmethyl, 2,3,4-trifluoro-phenylmethanesulfonylmethyl, 2,3,5-trifluoro-phenylmethanesulfonylmethyl, 2,5,6-trifluoro-phenylmethanesulfonylmethyl, 2-chloro-5-trifluoromethylphenylmethanesulfonylmethyl, 2-methyl-propane-1-sulfonyl, 2-fluoro-3-trifluoromethylphenylmethanesulfonylmethyl, 2-fluoro-4-trifluoromethylphenylmethanesulfonylmethyl, 2-fluoro-5-trifluoromethylphenylmethanesulfonylmethyl, 4-fluoro-3-trifluoromethylphenylmethanesulfonylmethyl, 2-methoxy-phenylmethanesulfonylmethyl, 3,5bis-trifluoromethyl-phenylmethanesulfonylmethyl, 4-difluoromethoxy-phenylmethanesulfonylmethyl, 2-difluoromethoxy-phenylmethanesulfonylmethyl, 3-difluoromethoxy-phenylmethanesulfonylmethyl, 2,6-dichloro-phenylmethanesulfonylmethyl, biphenyl-4-ylmethanesulfonylmethyl, 3,5-dimethyl-isoxazol-4-ylmethanesulfonylmethyl, 5-chloro-thiophen-2-ylmethanesulfonylmethyl, 2-[4-(1,1-Difluoro-methoxy)-benzenesulfonyl]-ethyl, 2-[2-(1,1-Difluoro-methoxy)-benzenesulfonyl]-ethyl, 2-[3-(1,1-Difluoro-methoxy)-benzenesulfonyl]-ethyl, 2-(4-trifluoromethoxy-benzenesulfonyl)-ethyl, 2-(3-trifluoromethoxy-benzenesulfonyl)-ethyl, 2-(2-trifluoromethoxy-benzenesulfonyl)-ethyl, (cyanomethyl-methyl-carbamoyl)-methyl, butyl, biphenyl-3-ylmethyl, 2-oxo-2-pyrrolidin-1-yl-ethyl, 2-benzenesulfonyl-ethyl, isobutylsulfanylmethyl, 2-phenylsulfanyl-ethyl, cyclohexylmethanesulfonylmethyl, 2-cyclohexyl-ethanesulfonyl, benzyl, naphthalen-2-yl, benzylsulfanylmethyl, 2-trifluoromethyl-benzylsulfanylmethyl, 5-bromo-thiophen-2-ylmethyl phenylsulfanyl-ethyl and cyclopropylmethanesulfonylmethyl.

5. The compound of claim 4 in which R⁴ is selected from phenylamino, benzylamino, 4-phenoxy-phenylamino, phenethylamino, 3-phenyl-propylamino, morpholin-4-yl, cyclohexylamino, naphthalen-1-ylmethyl-amino, pyridin-3-ylamino, 6-methoxy-pyridin-3-ylamino, diisobutylamino, 4-nitro-benzylamino, 2-thiophen-2-yl-ethylamino, 3-phenoxy-phenylamino, cyanomethyl-amino, (pyridin-3-ylmethyl)-amino, 5,6,7,8-tetrahydro-naphthalen-1-ylamino, 2-pyridin-2-yl-ethylamino, 2,3-dihydro-indol-1-yl, 3,4-dihydro-1H-isoquinolin-2-yl, cyclohexylmethyl-amino, 2-methoxy-benzylamino, 1-phenyl-ethylamino, (pyridin-4-ylmethyl)-amino, benzyl-methyl-amino, 3-nitro-benzylamino, 4-methoxy-phenylamino, 3-carbamoyl-phenylamino, 4-carbamoyl-phenylamino, (tetrahydro-furan-2-ylmethyl)-amino, 3,4-dihydro-2H-quinolin-1-yl, dimethylamino, butylmethylamino, diisopropylamino, propylmethylamino, 1-(benzooxazole-2-carbonyl)-propylamino and isobutylmethylamino.

6. The compound of claim 5 selected from the group consisting of: 2-butyl-N-cyanomethyl-N′-phenyl-malonamide; N-[1-(Benzooxazole-2-carbonyl)-3-phenyl-propyl]-N′-benzyl-2-cyclohexylmethyl-malonamide; 2-(2-benzenesulfonyl-ethyl)-N-[(S)-1-(1-benzooxazol-2-yl-methanoyl)-pentyl]-N-benzyl-malonamide; N,N′-bis-[(S)-1-(1-benzooxazol-2-yl-methanoyl)-propyl]-2-cyclohexylmethyl-malonamide; and the N-oxide derivatives, prodrug derivatives, protected derivatives, individual isomers and mixtures of isomers thereof, and the pharmaceutically acceptable salts and solvates of such compounds and the N-oxide derivatives, prodrug derivatives, protected derivatives, individual isomers and mixtures of isomers thereof.

7. A pharmaceutical composition comprising a therapeutically effective amount of a compound of claim 1 in combination with a pharmaceutically acceptable excipient.

8. A method for treating a disease in an animal in which inhibition of Cathepsin S can prevent, inhibit or ameliorate the pathology and/or symptomology of the disease, which method comprises administering to the animal a therapeutically effective amount of compound of claim 1 or a N-oxide derivative or individual isomer or mixture of isomers thereof, or a pharmaceutically acceptable salt or solvate of such compounds and the N-oxide derivatives, prodrug derivatives, protected derivatives, individual isomers and mixtures of isomers thereof.

9. A process for preparing a compound of Formula I:
(A) reacting a compound of Formula 2: 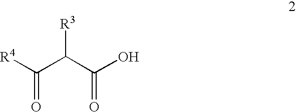
with a compound of formula NH₂CR¹R²X², in which R¹, R², R³, R⁴ and X² are as defined in the Summary of the Invention for Formula I; or
(B) reacting a compound of Formula 2 with a compound of Formula NH₂X³, in which R³, R⁴ and X³ are as described in the Summary of the Invention for Formula I; and
(C) optionally converting a compound of Formula I into a pharmaceutically acceptable salt;
(D) optionally converting a salt form of a compound of Formula I to non-salt form;
(E) optionally converting an unoxidized form of a compound of Formula I into a pharmaceutically acceptable N-oxide;
(F) optionally converting an N-oxide form of a compound of Formula I its unoxidized form;
(G) optionally resolving an individual isomer of a compound of Formula I from a mixture of isomers;
(H) optionally converting a non-derivatized compound of Formula I into a pharmaceutically prodrug derivative; and
(I) optionally converting a prodrug derivative of a compound of Formula I to its non-derivatized form.
